SEQUENCE LISTING (1 sequence)

SEQ ID NO: 1 - DNA, 21 bp, Artificial Sequence
  Description of Artificial Sequence Synthetic primer
tcgtcgtttttcggtcgtttt

(19) United States
(12) Patent Application Publication
(10) Pub. No.: US 2013/0273103 A1
(43) Pub. Date: Oct. 17, 2013

(54) POLYVALENT IMMUNOGEN

(71) Applicants: Hua-xin Liao, Durham, NC (US); Alan S. Lapedes, Los Alamos, NM (US); Barton F. Haynes, Durham, NC (US); Bette T. Korber, Los Alamos, NM (US)

(72) Inventors: Hua-xin Liao, Durham, NC (US); Alan S. Lapedes, Los Alamos, NM (US); Barton F. Haynes, Durham, NC (US); Bette T. Korber, Los Alamos, NM (US)

(73) Assignees: LOS ALAMOS NATIONAL SECURITY, LLC, Los Alamos, NM (US); DUKE UNIVERSITY, Durham, NC (US)

(21) Appl. No.: 13/876,830

(22) PCT Filed: Sep. 28, 2011
(86) PCT No.: PCT/US2011/001664

§ 371 Date: Mar. 28, 2013

Related U.S. Application Data
(60) Provisional application No. 61/387,392, filed on Sep. 28, 2010.

(51) Int. Cl. C07K 14/005

(52) U.S. Cl. CPC C07K 14/005
USPC 424/208.1; 530/395

(57) ABSTRACT

The present invention relates, in general, to HIV-1 and, in particular, to polyvalent immunogens suitable for use in inducing an immune response to HIV-1 in a patient, and to methods of identifying such immunogens. The invention further relates to methods of inducing an anti-HIV-1 immune response using such immunogens.

This application claims priority from U.S. Provisional Application No. 61/387,392, filed Sep. 28, 2010, the entire content of which is incorporated herein by reference.

This invention was made with government support under Grant No. U19 AI067854-05, awarded by the National Institutes of Health. The government has certain rights in the invention.

### TECHNICAL FIELD

The present invention relates, in general, to HIV-1 and, in particular, to polyvalent immunogens suitable for use in inducing an immune response to HIV-1 in a patient, and to methods of identifying such immunogens. The invention further relates to methods of inducing an anti-HIV-1 immune response using such immunogens.

### BACKGROUND

An effective HIV-1 vaccine ideally should target virus in the earliest stages of transmission, prior to dissemination and establishment of persistent infection (Haase, Nat. Rev. Immunol. 5:783-792 (2005), Hladik et al, Nat. Rev. Immunol. 8:447-457 (2008), Pope et al, Nat. Med. 9:847-852 (2003), Shattock et al, Nat. Rev. Microbiol. 1:25-34 (2003)). Results from the ‘Thai Trial’ RV144 of an experimental HIV-1 vaccine showed a decrease in virus acquisition of 31.2% (p=0.04) based on a modified intention-to-treat analysis (Rerks-Ngarm et al, NEJM 361: 2209-2220 (2009)). One candidate correlate of immunity is an antibody that was induced by the vaccine that, in a subset of subjects, inhibited HIV transmission at mucosal surfaces (Haynes et al, Current Opinion in HIV AIDS 5: 362-367 (2010)). Thus, to improve on the Thai RV144 trial, new procedures and algorithms need to be put in place to inform the choice of envelopes incorporated into the next generation of HIV-1 vaccines.

Recently, single genome amplification (SGA), direct sequencing, and a model of random virus evolution were employed to identify those viruses responsible for transmission and productive clinical infection in several cohorts with acute HIV-1 subtype A, B or C infection (Abrahams et al, J. Virol. 83:3556-3567 (2009), Haaland et al, PLoS Pathog. 5:e1000274 (2009), Keele et al, Proc. Natl. Acad. Sci. USA 105:7552-7557 (2008), Salazar-Gonzalez et al, J. Virol. 82:3952-3970 (2008), Salazar-Gonzalez et al, J. Exp. Med. 205:1273-1289 (2009)) and in Indian rhesus macaques inoculated intra-rectally with SIVmac251 or SIVsmmE660 (Keele et al, J. Exp. Med. 206:1117-1134 (2009)). This experimental approach allows for the distinction of transmitted/founder viruses that differ by as little as a single nucleotide (Keele et al, Proc. Natl. Acad. Sci. USA 105:7552-7557 (2008), Keele et al, J. Exp. Med. 206:1117-1134 (2009)). SGA-direct sequencing also makes possible the identification of transmitted viral sequences in linked transmissions, thereby enabling the unambiguous tracking of viruses from donor to recipient across mucosal surfaces (Haaland et al, PLoS Pathog. 5:e1000274 (2009), Keele et al, J. Exp. Med. 206:1117-1134 (2009)), and the molecular cloning and analysis of those viruses actually responsible for productive clinical infection (Salazar-Gonzalez et al, J. Exp. Med. 205:1.273-1289 (2009)).

Considerable work has been done evaluating a number of recombinant envelopes from chronically infected subjects and these Envs have generated primarily neutralizing antibody responses against Tier 1 (easy-to-neutralize) HIV-1 isolates (rev. in Haynes and Montefiori, Exp. Rev. Vaccines 5:579-95 (2006); Mascola and Montefiori, Annu. Rev. Immunol. 28:433-44 (2010), Pal et al, Virology 348:341-53 (2006)). Recent immunogenicity of EnvS derived from acutely infected HIV subjects have not induced broad neutralizing antibodies (Blish et al, J. Virology 84:2573-84 (2009)). However, both the recent RV144 trial (Rerks-Ngarrn et al, NEJM 361: 2209-2220 (2009)) and the VAX 004 gp120 Glade B trial, while not inducing broad neutralizing antibodies, did induce high titers of Tier 1 neutralizing antibodies (Gilbert et al, JID 202:595-605 (2010)) and it may be these types of antibodies that were able to prevent virion or virus infected cell movement across mucosal epithelia (Haynes et al, Current Opinion in HIV AIDS 5:362-367 (2010), McElrath and Haynes, Induction of Immunity to Human Immunodeficiency Virus Type-1 By Vaccination, Immunity 33(4):542-554 (2010)).

The present invention results, at least in part, from studies designed to define the antigenicity and immunogenicity of a large panel of chronic, consensus and transmitted/founder envelopes and to develop algorithms for choosing optimal HIV Env combinations for inducing both high titered antibodies against Tier 1 (easy-to-neutralize) HIV strains but also to induce low levels of antibodies that, at low levels, neutralize some Tier 2 (more-difficult-to-neutralize) HIV strains.

### SUMMARY OF THE INVENTION

In general, the present invention relates to HIV-1. More specifically, the invention relates to polyvalent HIV-1 envelope immunogens suitable for use in inducing an immune response to HIV-1 in a patient, and to methods of identifying such immunogens. The invention further relates to methods of inducing an anti-HIV-1 immune response using such immunogens.

Objects and advantages of the present invention will be clear from the description that follows.

### BRIEF DESCRIPTION OF THE DRAWINGS

FIG. 1. Antigenicity and immunogenicity of transmitted/founder HIV envelope oligomers compared to chronic HIV envelopes.

FIG. 2. Choosing optimal polyvalent vaccines.

FIG. 3. The neutralizing antibody response to MN, the vaccinating strain; SF162, a Tier one virus, and the geometric mean titer of Tier 2 responses in the Vox 004 human trial, comparing neutralizing antibody responses in placebo vaccinated individuals (blue), HIV vaccinated infected (red), and vaccinated uninfected (green). The uninfected HIV vaccinated individuals had much higher levels of Tier 1 SF162 antibodies, and some had above background levels of Tier 2 responses, while the vaccinated individuals had low SF162 levels no Tier 2 activity. This suggests that such responses may be useful. Men and women were considered separately because women made higher responses overall, and no vaccinated infected women were included in the study for comparison.

### DETAILED DESCRIPTION OF THE INVENTION

Recombinant chronic and consensus envelope immunizations have shown limited breath of induced neutralizing antibodies. The RV144 Thai trial of ALVAC prime, gp120 B/E boost showed 31% vaccine efficacy, most prominent during the first 6 months after vaccination. One candidate RV144 trial correlate of protection is a short-lived antibody that prevented HIV-1 acquisition. In addition to chronic and consensus envelopes, transmitted/founder envelopes constitute the most biologically relevant targets of neutralizing antibodies.

The present invention relates to polyvalent immunogens suitable for use in inducing an immune response against HIV-1 and to methods of identifying such immunogens.

Briefly, a series of HIV-1 neutralization assays (such as the TZMb1 pseudovirus inhibition assay (Seaman et al, J. Virol. 84:1439-52 (2010))) provide titers of sera from immunized animals, such as guinea pigs, rabbits or rhesus macaques, that have been innoculated with different single members of a set of envelopes of interest and assayed against a panel of isolates of interest. Multiple animals, e.g., four, are each inoculated with the same immunogen. Multiple immunogens (envelopes) , e.g, twenty, from which a subset is to be chosen to form a polyvalent vaccine, can be used in any given assay. The geometric average of the neutralization titers across animals inoculated with the same immunogen are computed, separately for each isolate, to provide an average immunized animal response for each isolate. If more than a specified number of animals, e.g. two animals out of four, contributing to such an average have titers over a given threshold value, then the computed average is accepted as a robust summary of the average immunized animal response to that isolate. Otherwise the computed average is replaced with a background value. Next, a vaccine valency is chosen, e.g., valency equal to six. Given, e.g., twenty immunogens then there are 20-choose-6 or 38,760 combinations of immunogens that are potential polyvalent vaccine candidates. Assuming that the effect of a polyvalent vaccine is roughly the sum of its components, then the titer of any polyvalent vaccine candidate with an isolate can be computed from the individual assay data. Each of the polyvalent vaccine candidates (combination of immunogens) is scored by a method described next, and the combination(s) that score highest are chosen.

The score rewards high overall mean titer across isolates of a polyvalent vaccine candidate (rewards OverallMeanTiter), penalizes polyvalent vaccine candidates that do not have at least one component with above-threshold titers on members of the panel of isolates (penalizes IsolatesNotCovered), and rewards polyvalent vaccine candidates that have more than one vaccine component with above threshold titer on isolates (rewards AverageDepth). OverailMeanTiter is the overall mean titer across isolates of the polyvalent vaccine candidate. IsolatesNotCovered is the number of isolates for which no member of the polyvalent vaccine has an above-threshold titer. AverageDepth is the number of vaccine components that have above-threshold titers on an isolate, averaged across isolates. The score =OverallMeanTiter-(A*IsolatesNotCovered)+(B*AverageDepth), where A and B are coefficients which can be determined by a simple grid search. For example, A and B may be initially set to zero and a set of vaccine candidates identified that have high OverallMeanTiter and also have AverageIsolatesNotCovered=0 (if the latter exists). Then B can be increased to find the set that has greater AverageDepth. If no initial set with AverageIsolatesNotCovered=0 is found, then A can be increased to attempt to find a set of polyvalent vaccine candidates that cover all isolates. If successful, the B can be subsequently increased to attempt to add Depth. Immunogen combinations that place at or near the top of the list of scores are candidates for polyvalent vaccines.

Using the above-identified process, certain high-scoring polyvalent immunogens have been identified. In one embodiment, the choice of polyvalent immunogens is 3-valent and comprises gp140 Envs: (B.0040, B.6240, C.089), also (B.0040, B.6240, B.62357), also (CON.S, B.0040, B.6240), also (CON.S, B.0040, C.089). In another embodiment, the polyvalent immunogen is 6-valent and comprises gp140 Envs: (CON.S, B.63521, B.0040, B.62357, B.6240, C.089), also (CON.S, Al .con.env.03.140CF, C.con.env.03.140CF, CON.T, B.0040, C.089), for induction of high titers of Tier 1 antibodies, a combination of 1086.0 and group M consensus Env CON-S can be used.

It has recently been discovered, by revisiting data collected by others in the Vax004 large human HIV vaccine trial (that was not protective), that very high levels of heterologous Tier 1 antibodies (SF612), as well as any above threshold Tier 2 antibody responses were highly enriched in uninfected versus infected vaccinees. Given that the trial did not show protection overall, this could not be shown to be a correlate of protection but does suggest that these were useful responses in the small fraction of vaccines that made them. It also suggests that the frequency of high Tier 1 or Tier 2 responding individuals may be a critical measure of success. Thus, these features can be weighted among the polyvalent vaccine selection strategies, and some combinations that feature Con S as well as Tier 2 responders can be included to maximize complemenatarity of potentially beneficial responses.

The present invention also relates to a method of inducing the production in a subject (e.g., a human subject) of an immune response against HIV-1. The method comprises administering to the subject a polyvalent immunogen identifiable, for example, using the process described above in an amount and under conditions such that an immune response against HIV-1 is produced..

In accordance with the invention, the polyvalent immunogen can be used in a DNA prime, gp120 or gp140 protein boost, or can be used alone as a protein prime and boost. The Env immunogens can be present in a liposome, for example, with one or more adjuvants. Alternatively, the immunogen protein can be otherwise formulated with one or more adjuvants. Other vectors such as recombinant mycobacteria, recombinant vaccinia or vaccinia derivatives, as well as recombinant adenoviruses can be used to deliver the polyvalent Env immunogen, as primes and boosts, both with or without recombinant protein boosts.

Suitable adjuvants include, for example, monophosphorylipid A (MPL-A) (Avanti Polar Lipids, Alabaster, AL), a TLR 9 agonist, such as oCpGs 10103 (5′-TCGTCGTTTTTCGGTCGTTTT-3′) and R848 TLR 7 agonist (Enzo Life Sciences, Farmingdale, N.Y.). In addition, cytokine stimulators of B cell class switching, such as BAFF (BLYS) and/or APRIL (He et al, Immunity 26:812-26 (2007); Cerutti and Rescigno, Immunity 28: 740-50 (2008)) can be incorporated into the liposomes for optimal B cell stimulation.

Liposomes suitable for use in the invention include, but are not limited to, those comprising POPC, POPE, DMPA (or sphingomyelin (SM)), lysophosphorylcholine, phosphatidylserine, and cholesterol (Ch). While optimum ratios can be determined by one skilled in the art, examples include POPC:POPE (or POPS):SM:Ch or POPC:POPE (or POPS):DMPA:Ch at ratios of 45:25:20:10.

Alternative formulations of liposomes that can be used include DMPC (1,2-dimyristoyl-sn-glycero-3-phosphocholine) (or lysophosphorylcholine), cholesterol (Ch) and DMPG (1,2-dimyristoyl-sn-glycero-3-phoshpho-rac-(1-glycerol) formulated at a molar ratio of 9:7.5:1 (Wassef et al, ImmunoMethods 4:217-222 (1994); Alving et al, G. Gregoriadis (ed.), Liposome technology 2^{nd} ed., vol. III CRC Press, Inc., Boca Raton, Fla. (1993); Richards et al, Infect. Immun. 66(6):285902865 (1998)). The above-described lipid compositions can be complexed with lipid A and used as an immunogen to induce antibody responses against phospholipids (Schuster et al, J. Immunol. 122:900-905 (1979)). A preferred formulation comprises POPC:POPS:Ch at ratios of 60:30:10 complexed with lipid A according to Schuster et al, J. Immunol. 122:900-905 (1979). The optimum ratio of peptide to total lipid can vary, for example, with the peptide and the liposome.

A variety of adjuvants can be used in the present invention (including those noted above). The immunogens and conjugates described above can be formulated with, and/or administered with, adjuvants such as squalene-based adjuvants (Kaldova, Biochem. Biophys. Res. Communication, Dec. 16, 2009 E-pub ahead of print) and/or TLR agonists (e.g., a TRL 3, TRL 5, TRL4, TRL9 or TRL7/8 agonst, or combination thereof) that facilitate robust antibody responses (Rao et al, Immunobiol. Cell Biol. 82(5):523 (2004)). Other adjuvants that can be used include alum and Q521. Oligo CpGs in an oil emulsion such as Emulsigen (an oil in water emulsion) (Tran et al, Clin. Immunol. 109(3):278-287 (2003)) can also be used. Additional suitable adjuvants include those described in 11/302,505, filed Dec. 14, 2005, including the TRL agonists disclosed therein. (See also Tran et al, Clin. Immunol. 109:278-287 (2003), US Appln Nos. 20030181406, 20040006242, 20040006032, 20040092472, 20040067905, 20040053880, 20040152649, 20040171086, 20040198680, 200500059619). Immune response enhancing TLR ligands, such as Lipid A, oligo CpG and R-848 can be formulated individually or in combination into liposomes that have HIV-1 Env conjugated in them.

The mode of administration of the HIV-1 protein/polypeptide/peptide, or encoding sequence, can vary with the immunogen; the patient and the effect sought, similarly, the dose administered. Typically, the administration route will be intramuscular, intravenous, intraperitoneal or subcutaneous injection. Additionally, the formulations can be administered via the intranasal route, or intrarectally or vaginally as a suppository-like vehicle. Generally, the liposomes are suspended in an aqueous liquid such as normal saline or phosphate buffered saline pH 7.0. Optimum dosing regimens can be readily determined by one skilled in the art. The immunogens are preferred for use prophylactically, however, their administration to infected individuals can reduce viral load.

Certain aspects of the invention are described in greater detail in the non-limiting Examples that follow (see also Maksyutov et al, J. Clin. Virol. Dec; 31 Suppl 1:S26-38 (2004), Haynes et al, Science 308:1906 (2005), U.S. Pat. No. 7,611,704, U.S. application Ser. No. 11/812,992, filed Jun. 22, 2007, U.S. application Ser. No. 11/785,077, filed Apr. 13, 2007, PCT/US2006/013684, filed Apr. 12, 2006, PCT/USO4/30397 (WO2005/028625), WO 2006/110831, WO 2008/127651, WO 2008/118470, U.S. Published Application Nos. 2008/0031890 and 2008/0057075, U.S. application Ser. No. 11/918,219, filed Dec. 22, 2008, U.S. Provisional Application No. 61/282,526, filed Feb. 25, 2010, U.S. Provisional Application No. 61/322,725, filed Apr. 9, 2010, U.S. Provisional Application No. 60/960,413, filed Feb. 28, 2007, and U.S. Provisional Application Nos. 61/166,625, 61/166,648 and 61/202,778, all filed Apr. 3, 2009, the entire contents of which are incorporated herein by reference). (See also http://www.hiv.lanl.gov/content/sequence/HIV/mainpage.html.)

### EXAMPLE 1

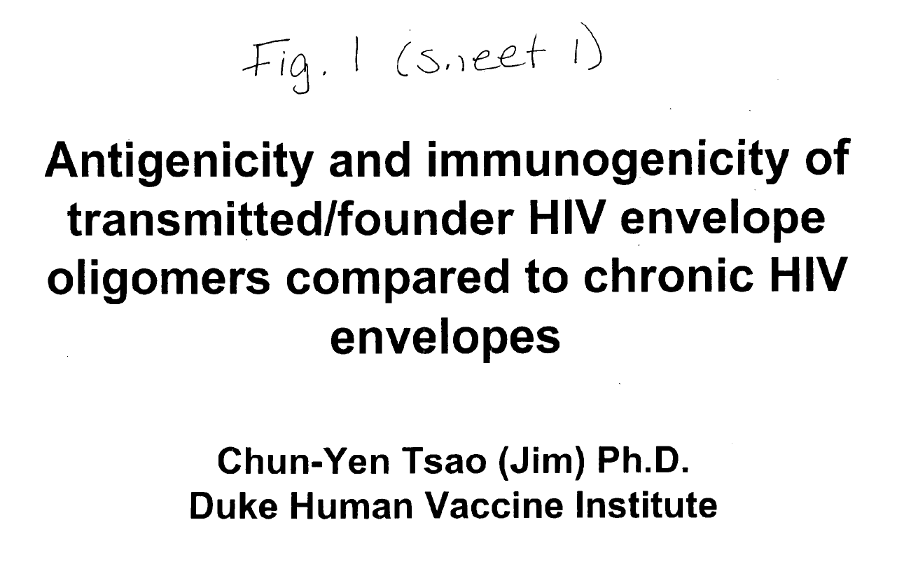

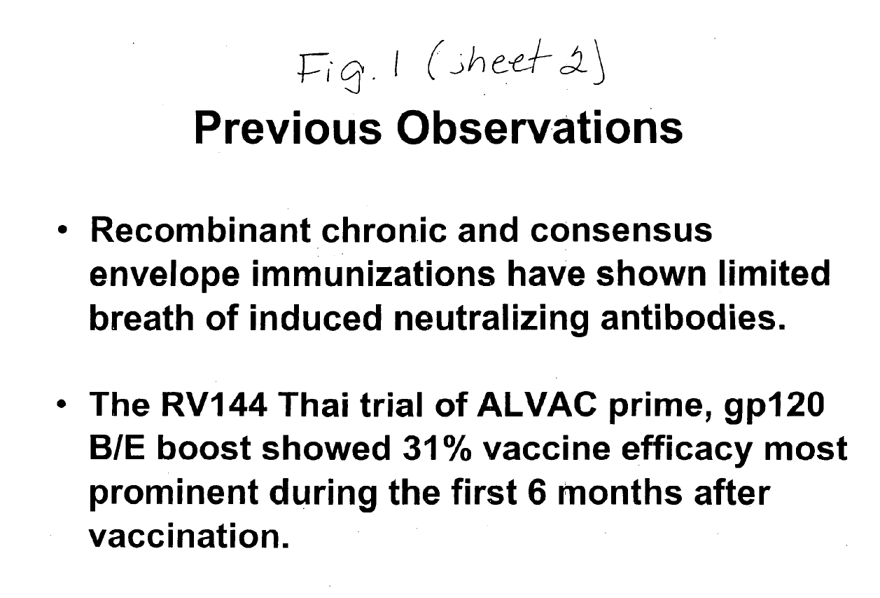

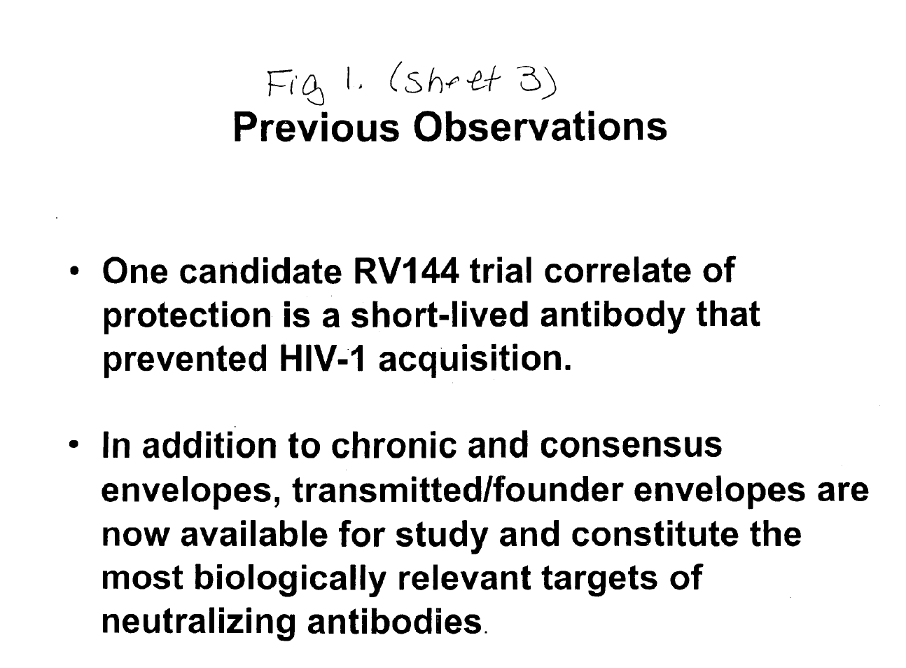

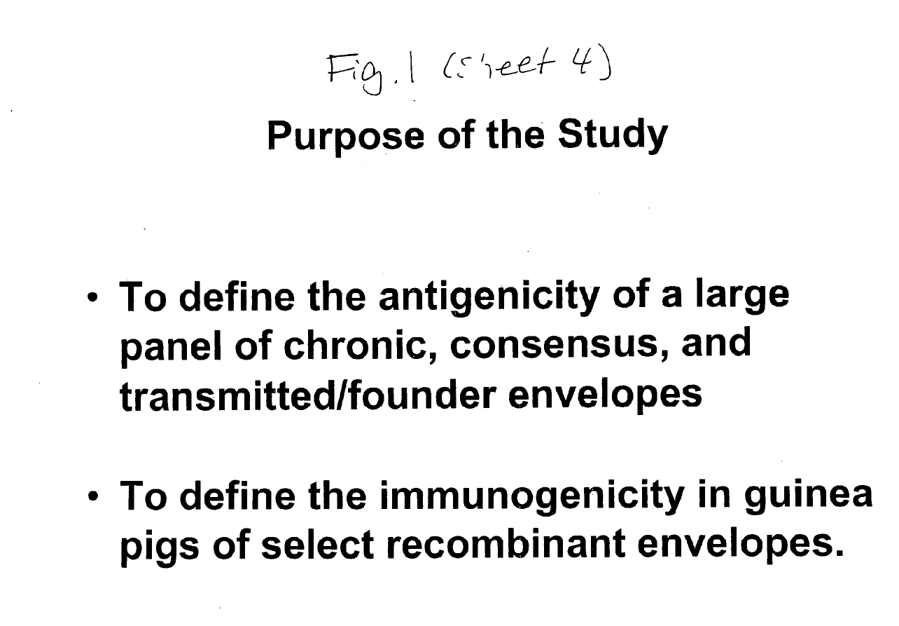

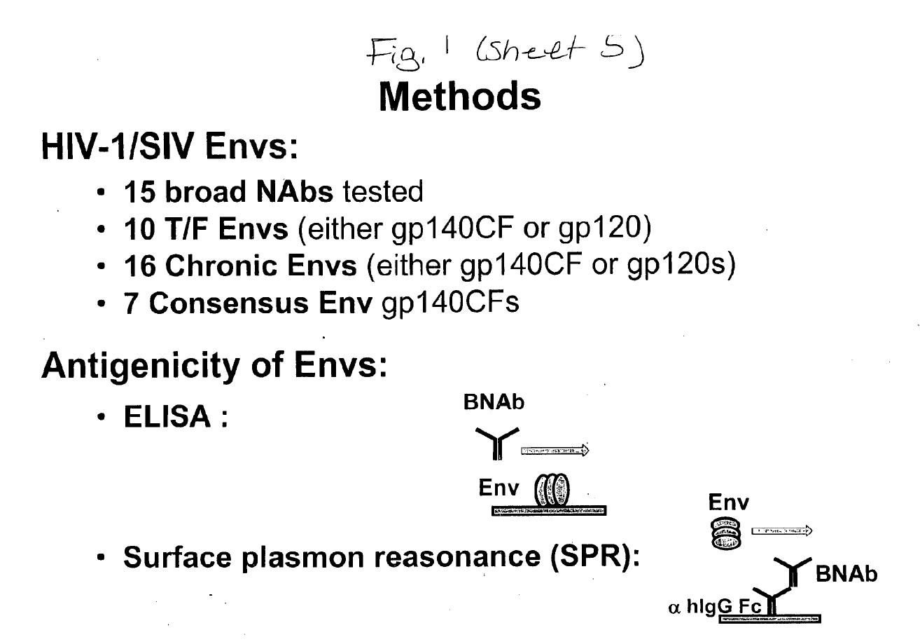

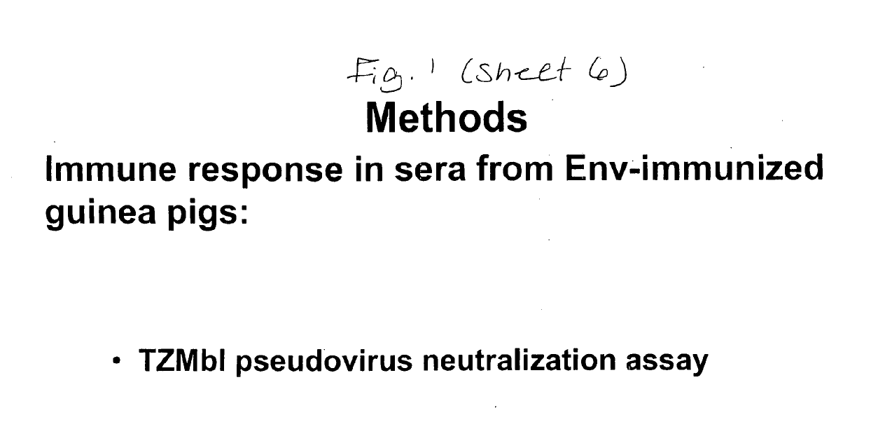

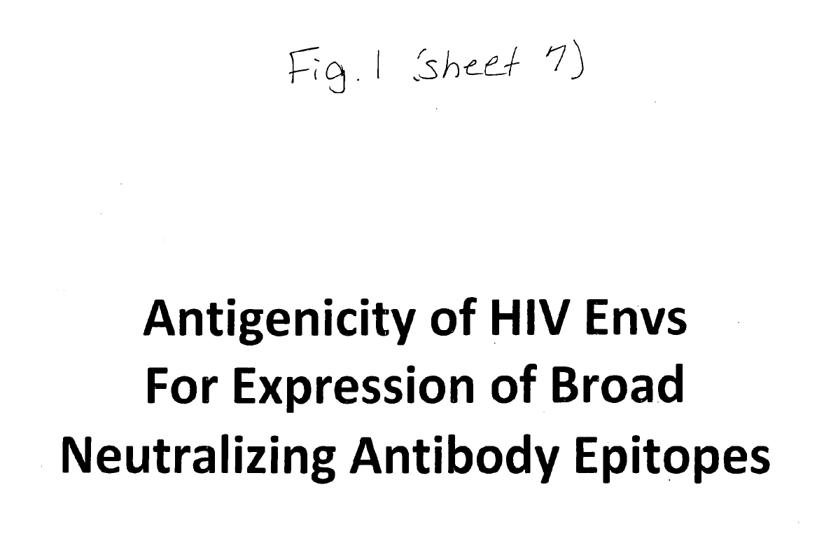

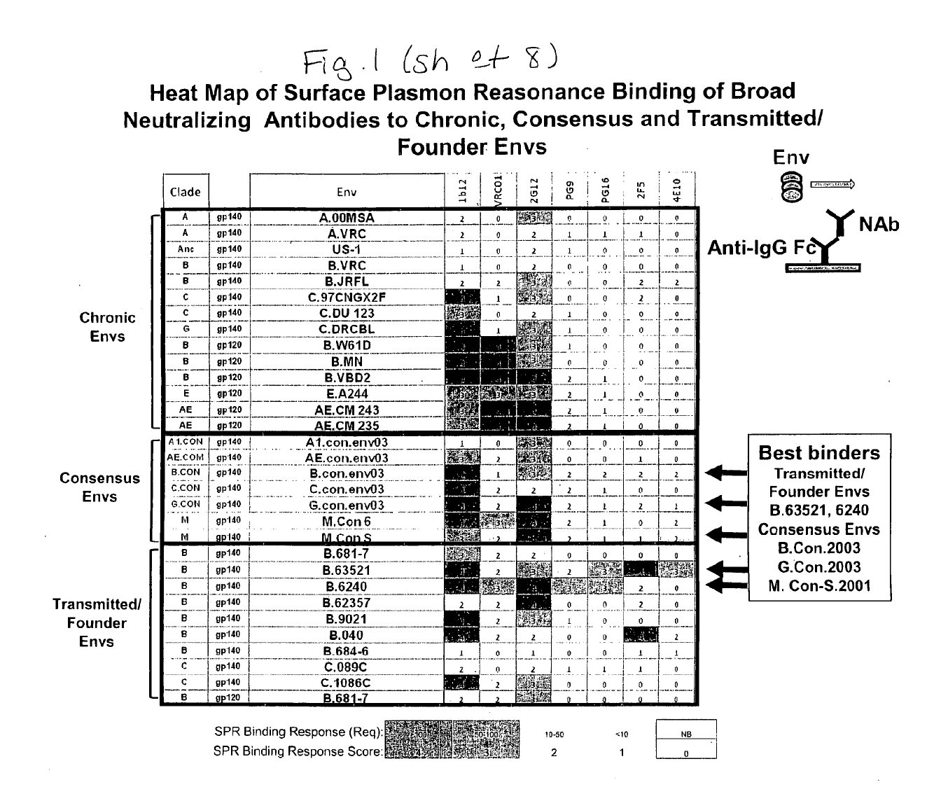

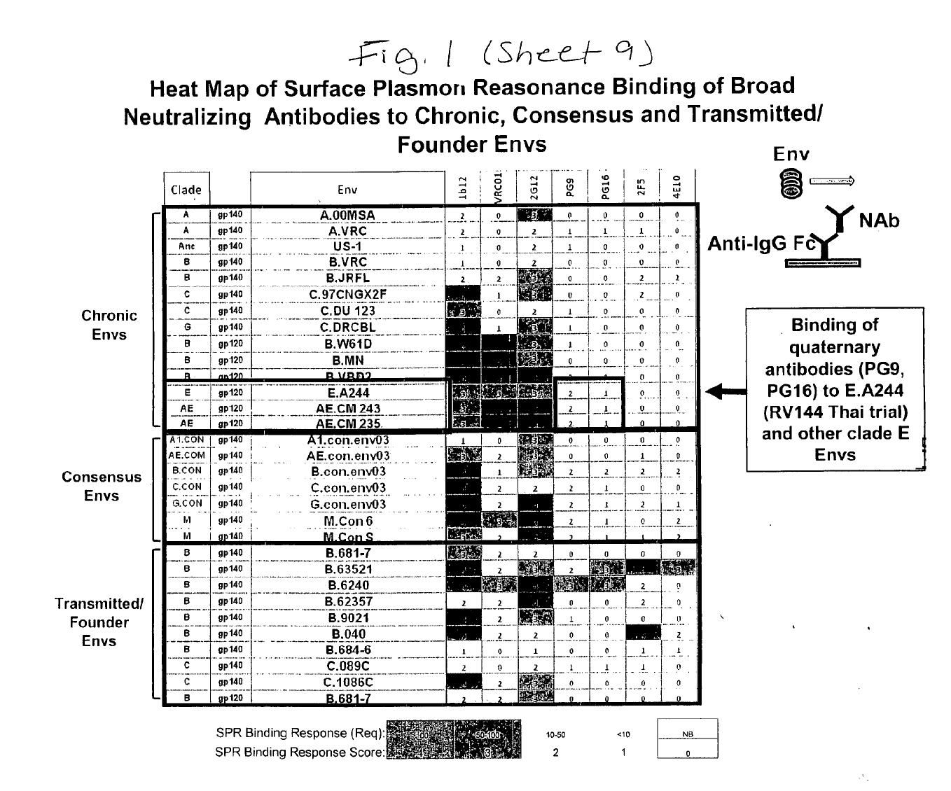

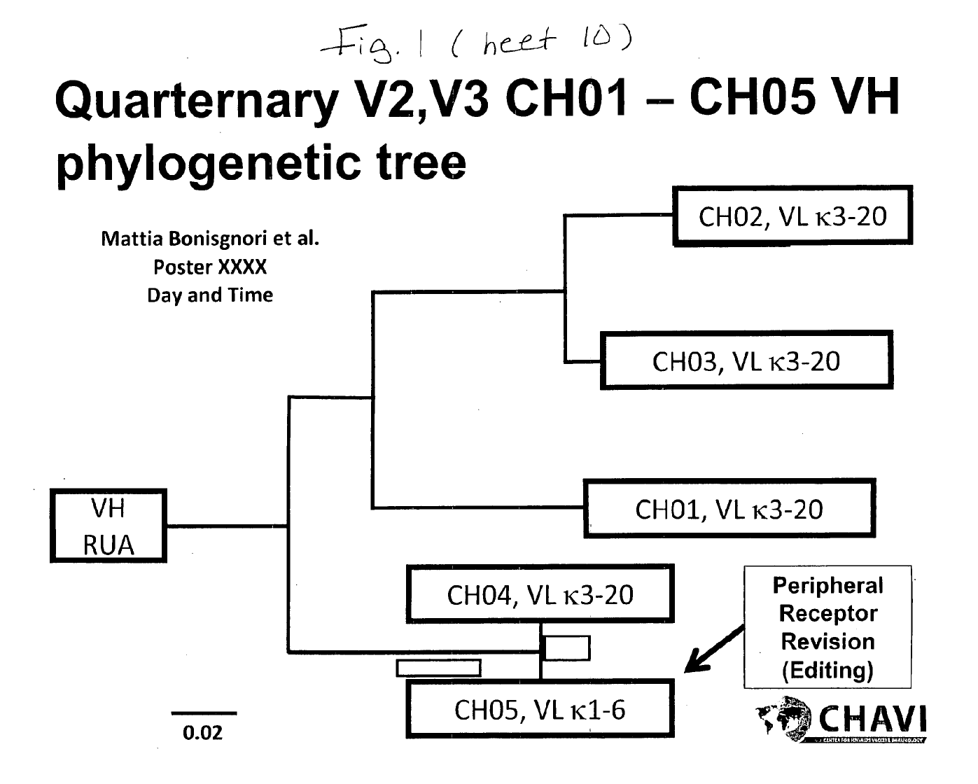

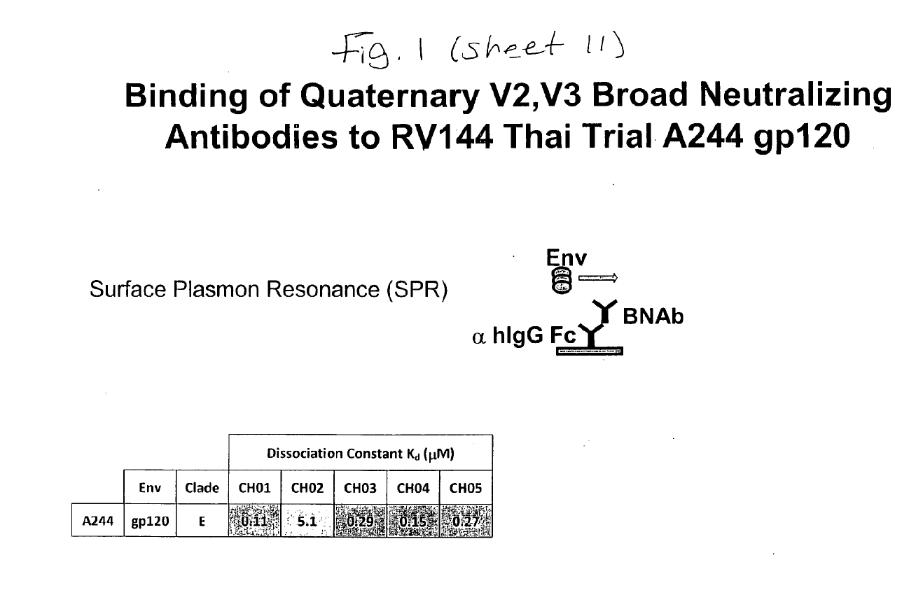

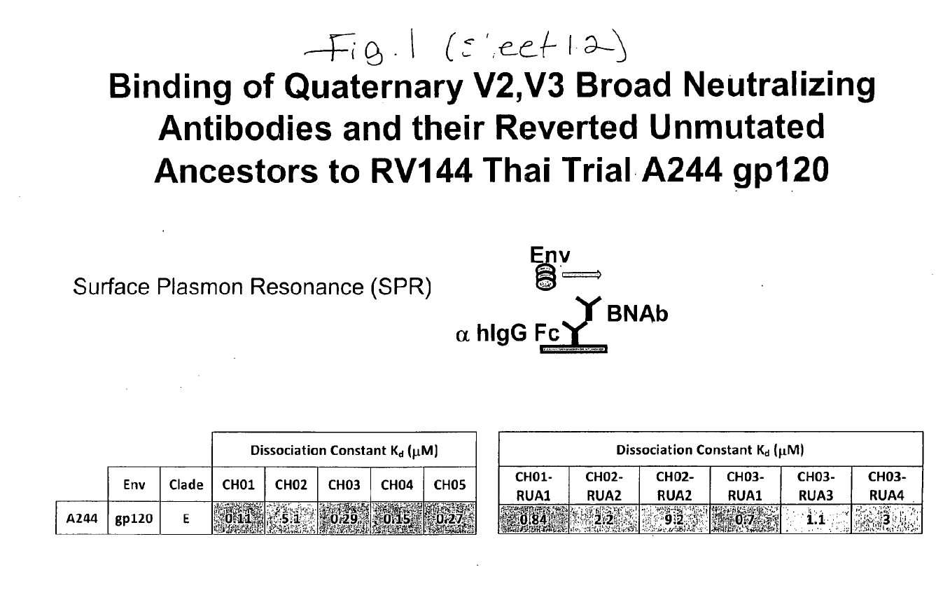

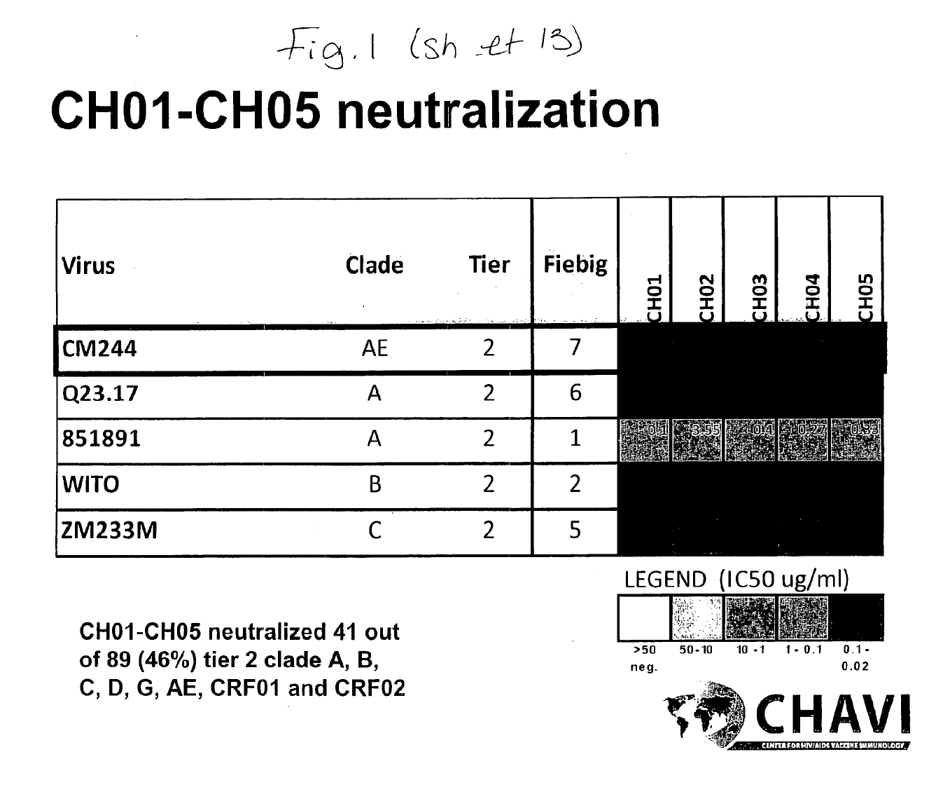

Fifteen broadly neutralizing antibodies were tested on 10 transmitted/founder envelopes, 15 chronic envelopes, and 7 consensus recombinant envelopes (see FIG. 1). The antigenicity of these envelopes for binding neutralizing antibodies was first determined by ELISA with envelopes bound on the plate surface. The antigenicity of these envelopes was also determined by Surface Plasmon Resonance (SPR) in which envelopes flowed over neutralizing antibodies on the SPR chip.

The immune response of Env-immunized guinea pigs was tested in TZMbI pseudovirus neutralization assays (Perez et al, J. Virol. 83(15):7397 (2009)) performed with a panel of 36 tier 1 and tier 2 pseudoviruses. SPR assays demonstrated that both AE group chronic envelopes and several consensus envelops have strong binding to the broad neutralizing antibodies. The best binders are now transmitted/founder envelope B.63521, B.6240, Consensus envelops,B.con.2003, G.con.2003, and M.Con-S.2001. (See sheet 8 of FIG. 1.) (See, for example, U.S. Provisional Application No. 61/282,526; for Transmitted Envs, see Keele et al, PNAS USA 105:7552-7 (2008) for Glade Bs and Abrahams et al, J. Virol 83:3556-67 (2009) for Glade Cs).

Interestingly, strong binding of RV 144 Thai trial Envelop E.A244 and other Glade AE envelopes to quaternary broad neutralizing antibodies PG9 and PG16 as well as to other broad neutralizing antibodies was observed. (See sheet 9 of FIG. 1.) Recently, 5 quaternary broad neutralizing antibodies have been isolated from a chronic HIV-1 infected patient. With an exception of CH05, all CH antibodies have V kappa 3-20 light chain. Their analysis has suggested a. possibility of peripheral receptor revision in the CH05 antibody. (See sheet 10 of FIG. 1.)

In SPR, CH01, CH03, CH04, CH05 showed strong binding reactivity to

A244 gp120, while CH02 showed a moderate binding reactivity to A244 gp120. Furthermore, reverted unmutated ancestors of CHOLCH02 and CH03 quarternary antibodies also bind to A244 gp120 in SPR with relative weaker dissociation constants. (See sheet 11 of FIG. 1.) CH antibodies neutralized ˜45% of Tier 2 viruses tested including CM244 AE_{—}01 recombinant. The CM244 isolate (envelope designated A244) was also neutralized by three CH01-03 RUAs.

In addition to the antigenicity studies of these envelopes, guinea pigs have been immunized with 5 chronic, 8 consensus and 7 transmitted/founder gp 140 envelope oligomers. Immunization was effected by intramuscular injection with oil in water emulsion and type B oligo-CpGs adjuvant. Sera from these immunized guinea pigs were assayed against 36 tier 1 and tier 2 Glade A, B, C isolates. (See sheet 15 of FIG. 1.)

Sheet 16 of FIG. 1 shows .the gp140 env oligomers used to generate sera on the Y axis and the Tier 1 and Tier 2 pseudoviruses use to assay the sera on the X-axis.

In sheet 17 of FIG. 1, the black boxes show the chronic and consensus envelop sera and the red box shows that the chronic and consensus Envs induced primarily Tier 1 HIV isolate neutralization. The best inducers of Tier 1 neutralizing antibodies were group M consensus Env CON-S and the Transmitted/founder Env , C.1086. However immunization of transmitted/founder envelopes induced a neutralizing response against Tier 1 viruses as well as a weak neutralizing response to Tier 2 viruses. (See sheet 18 of FIG. 1.) When envelopes were captured by broad neutralizing antibodies in surface plasmon reasonance, transmitted/founder envelopes, B.63521 and B.6240 again were most antigenic, along with three consensus envelopes. (See U.S. Prov. Appln. No. 61/282,526.)

The criteria for choosing a polyvalent immunogen are set forth on sheet 19 of FIG. 1. Preferred 3-valent gp140 Envs include: (B.0040, B.6240, C.089), also (B.0040, B.6240, B.62357), also (CON.S, B.0040, B.6240), also (CON.S, B.0040, C.089). Preferred 6-valent gp140 Envs include: (CON.S, B.63521, B.0040, B.62357, B.6240, C.089), also (CON.S, Al .con.env.03.140CF, C.con.env.03.140CF, CON.T, B.0040, C.089). As shown on sheet 22 of FIG. 1, RV144 Thai trial, A244 gp120 reacted with most quaternary antibodies tested. RV144 Thai trial, A244 gp120 also reacted with all reverted unmutated ancestors (RUAs) tested of the CH01, CH02 and CH03 quaternary antibodies. Three out of six RUAs of these quaternary antibodies, neutralized the CM244 pseudovirus (A244).

In guinea pigs, vaccination with chronic or consensus envelops induced strong neutralization of Tier 1 pseduoviruses. Vaccination with transmitted/founder envelopes induced similar Tier 1 and weak Tier 2 neutralization breadth. (See sheet 23 of FIG. 1.)

The reactivity of the RV144 vaccine gp120, A244, with 7 quaternary human monoclonal antibodies and 6 of their RUAs was striking and suggests that the A244 gp120 is in a conformation similar to that in the native trimer—regarding the epitope bound by V2, V3 quaternary monoclonal antibodies.

### EXAMPLE 2

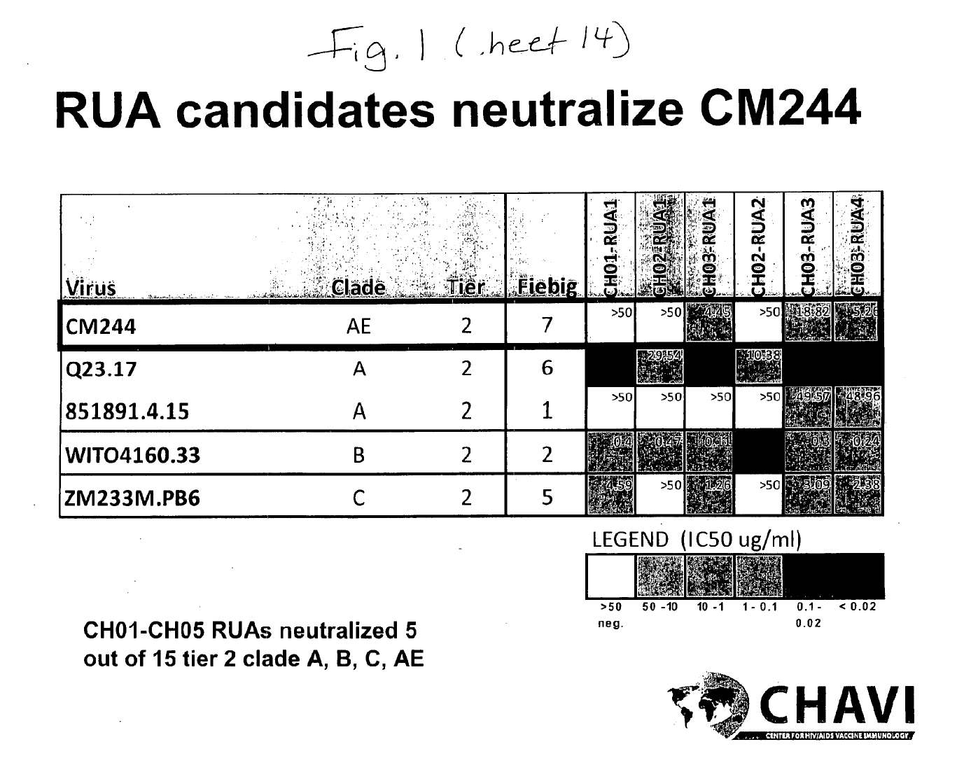

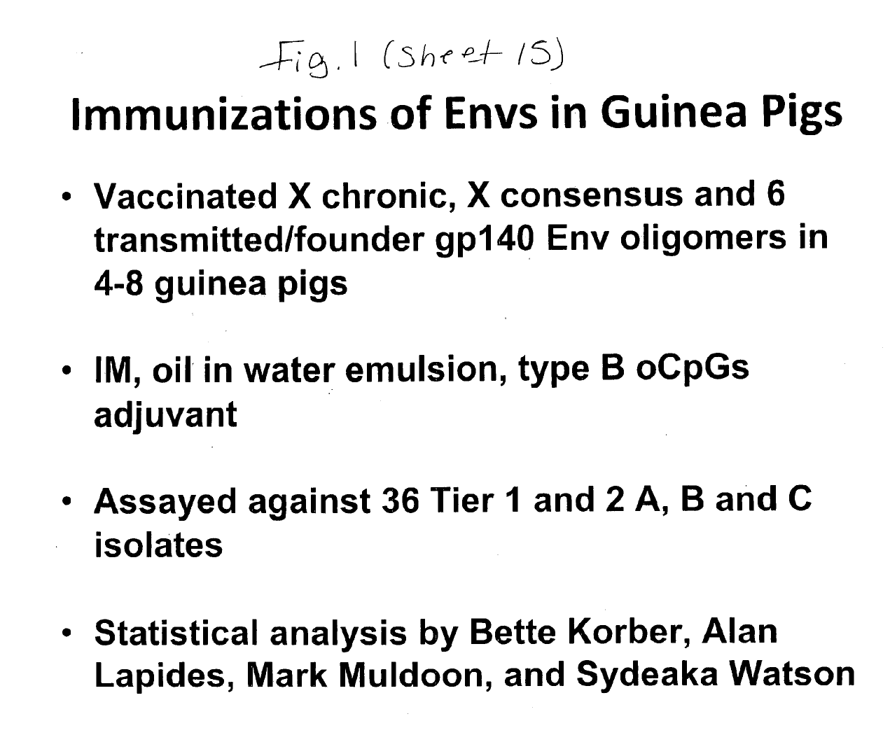

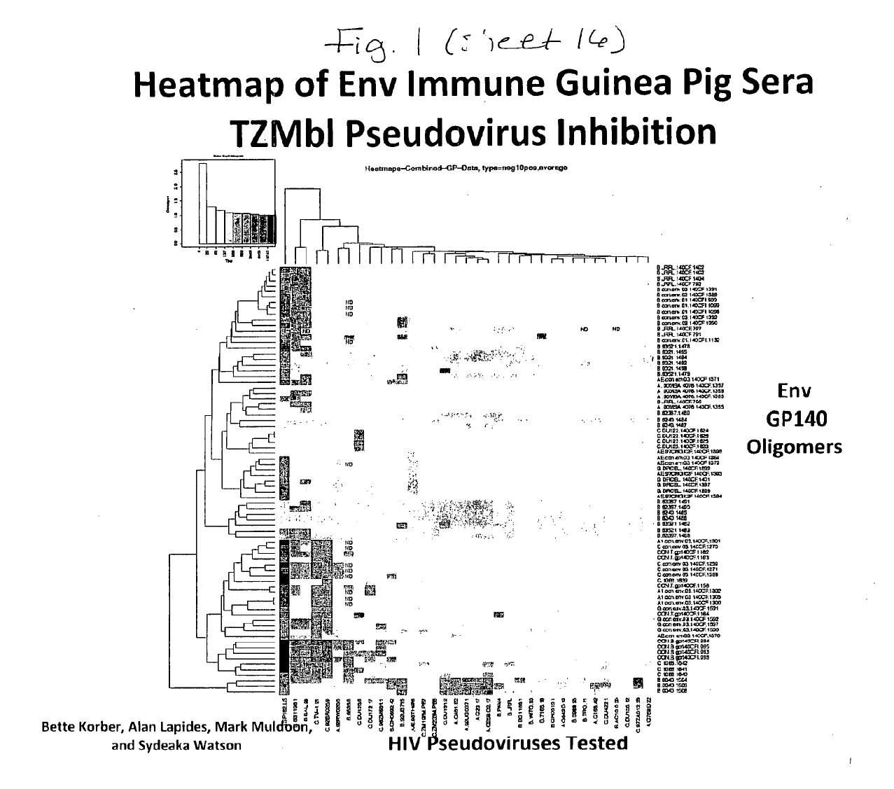

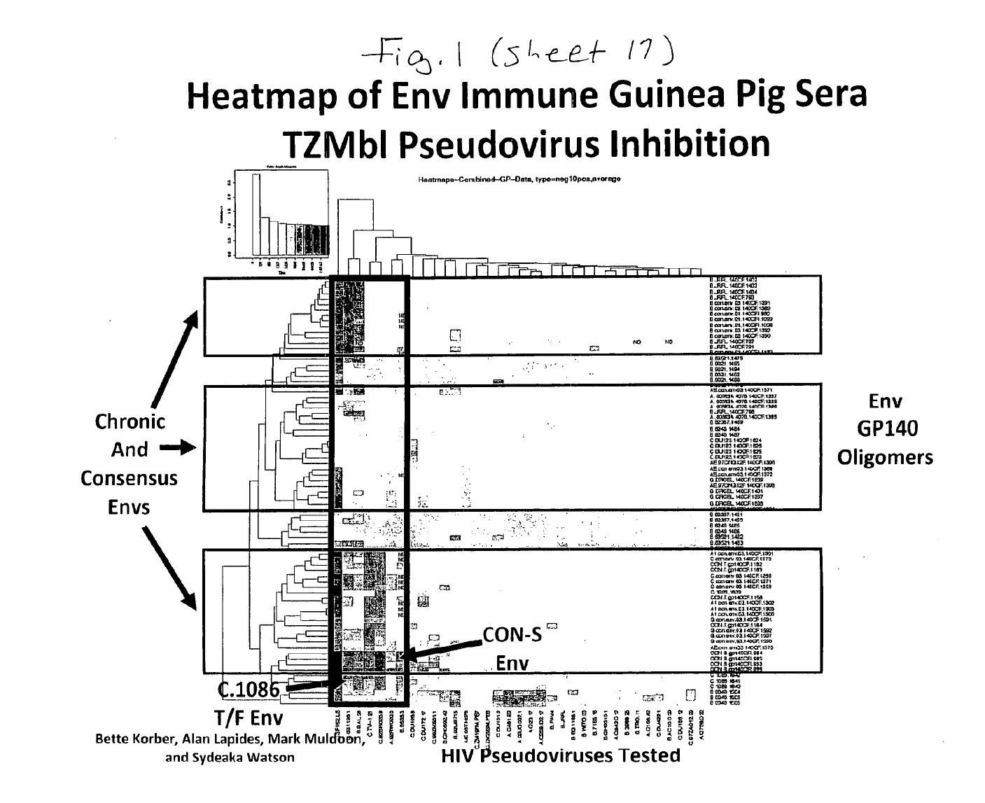

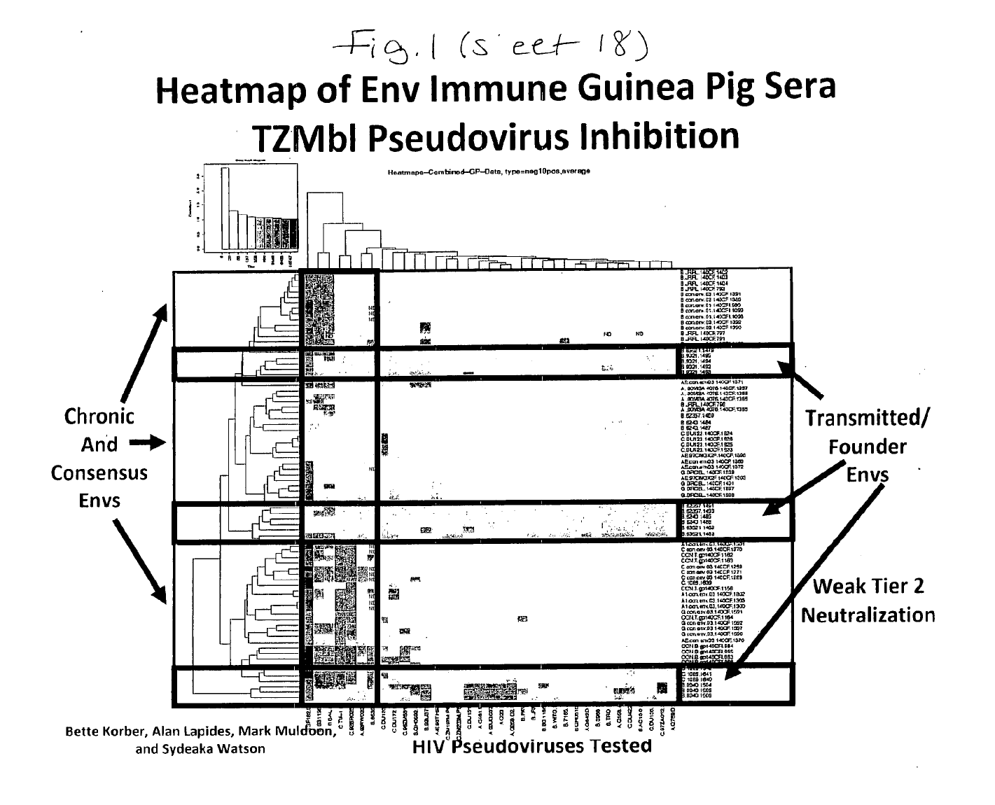

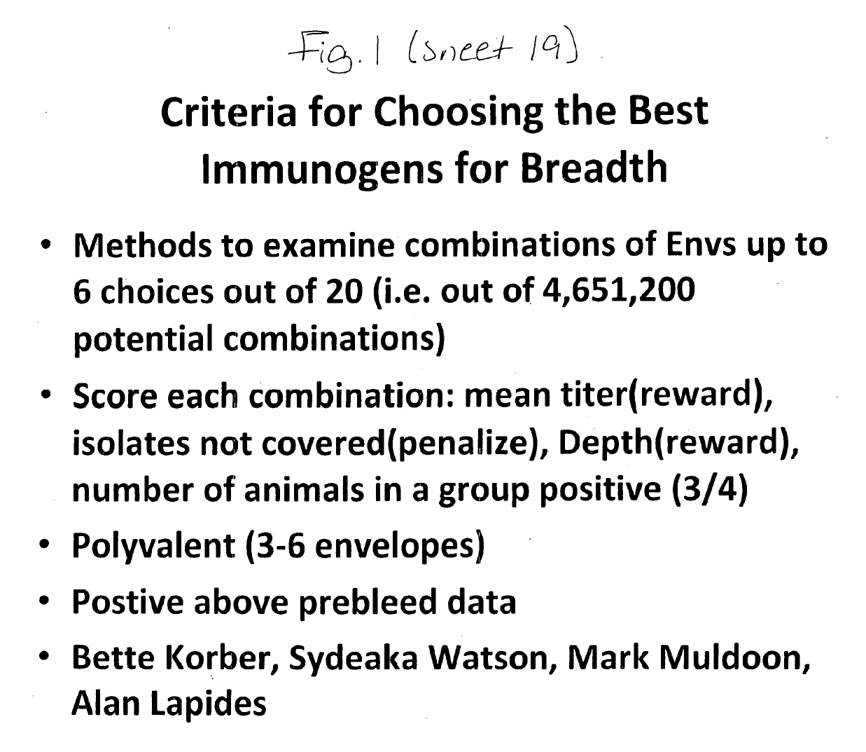

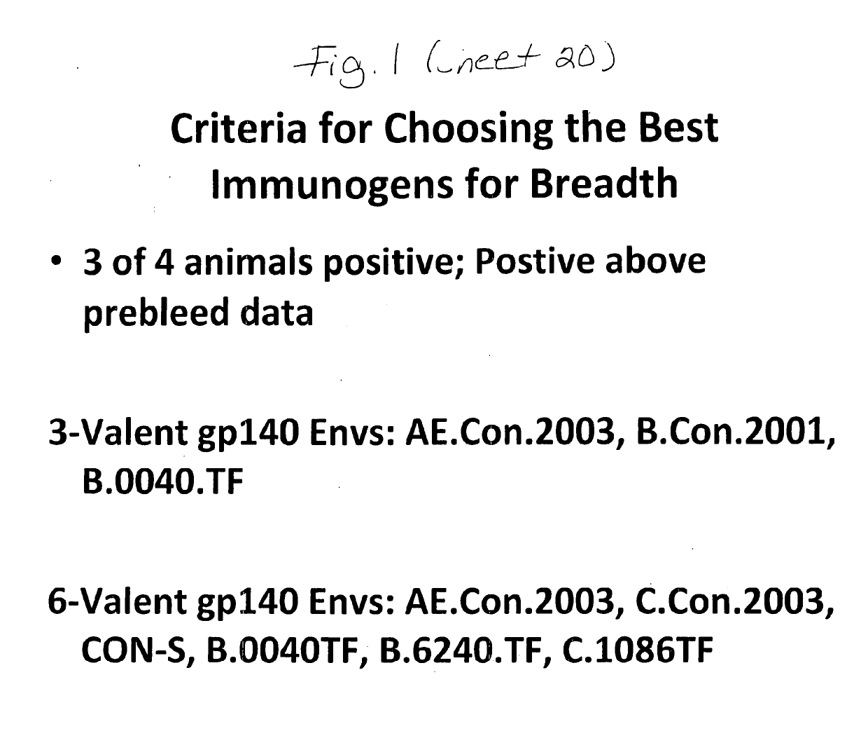

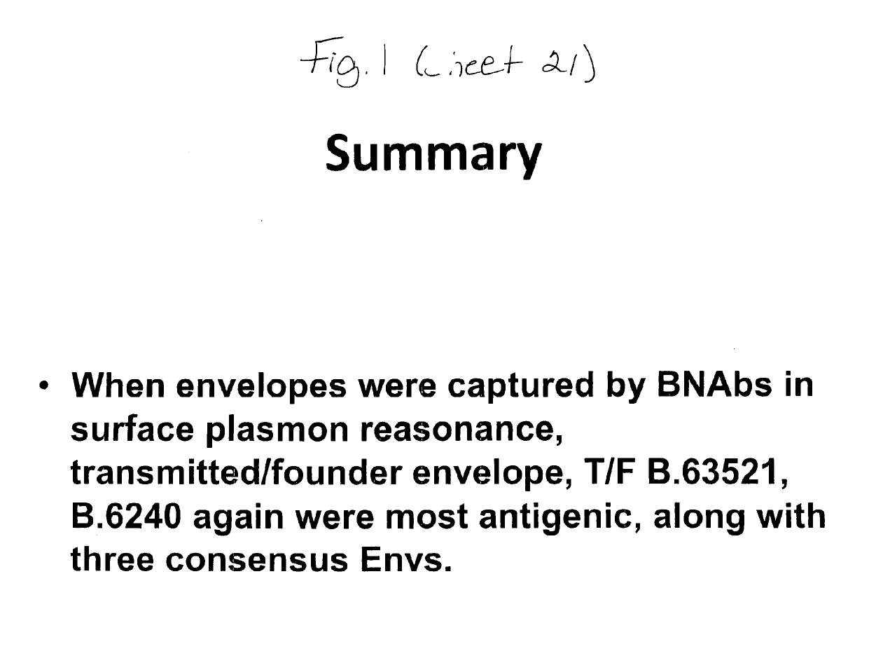

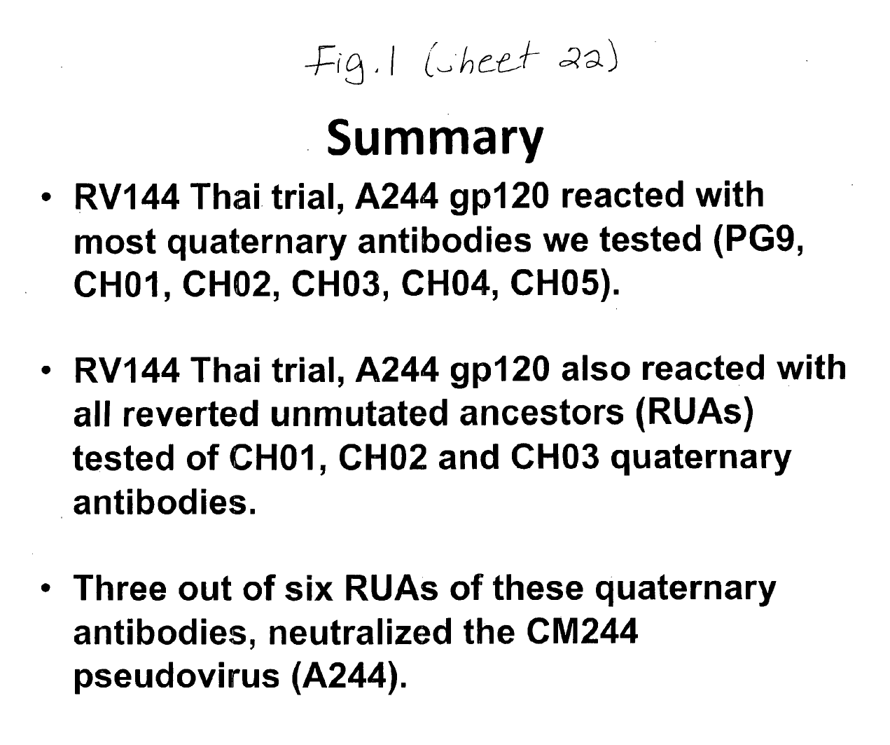

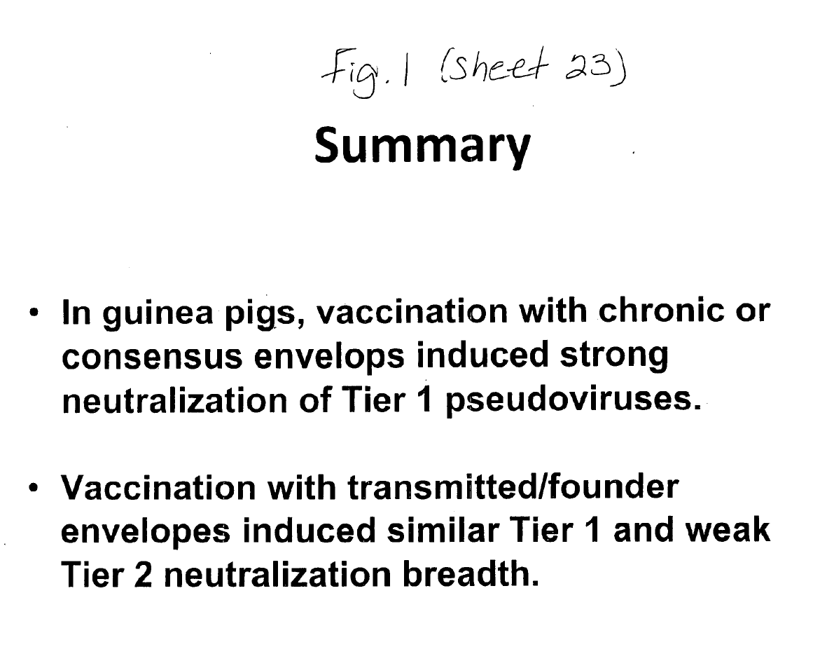

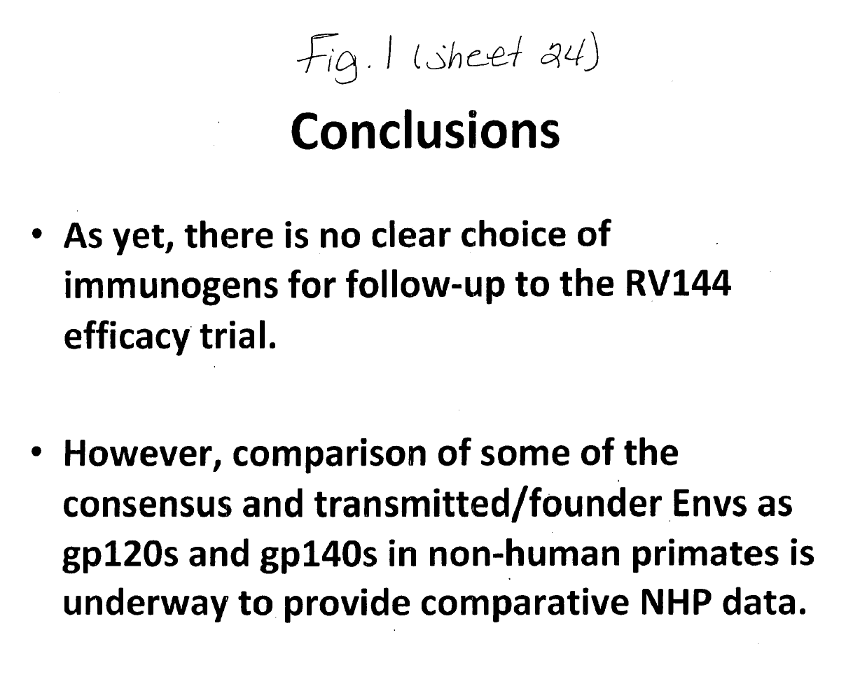

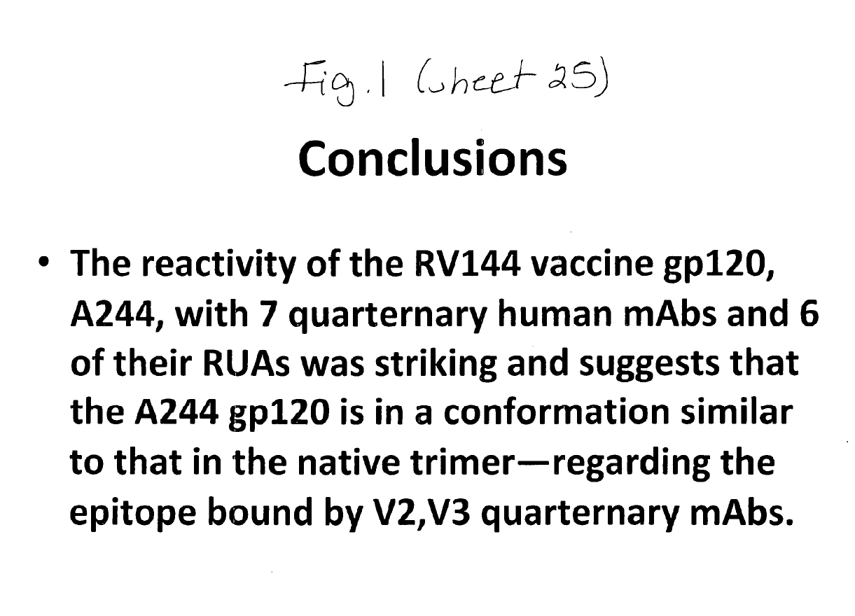

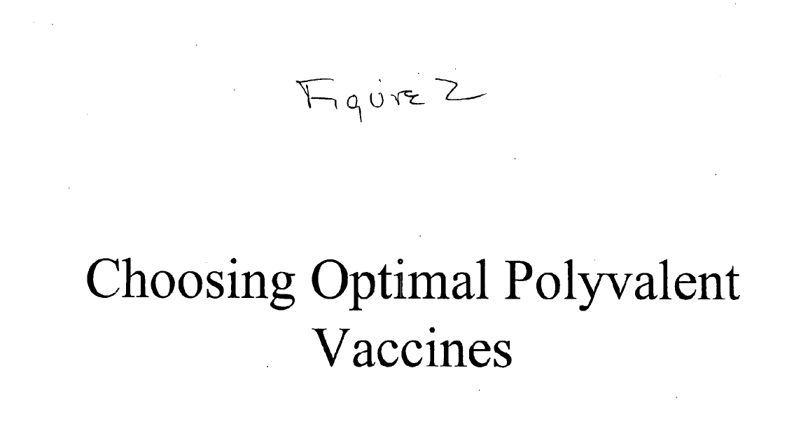

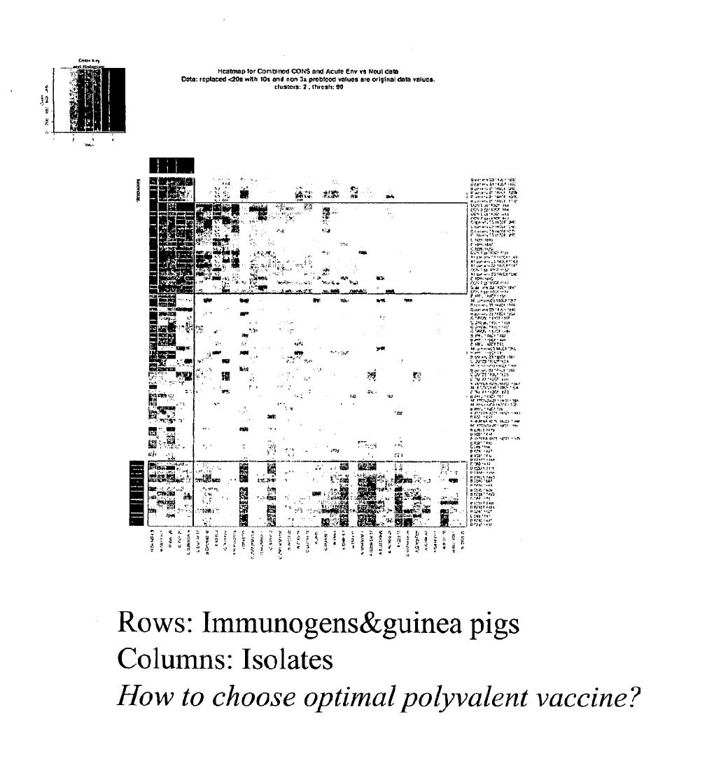

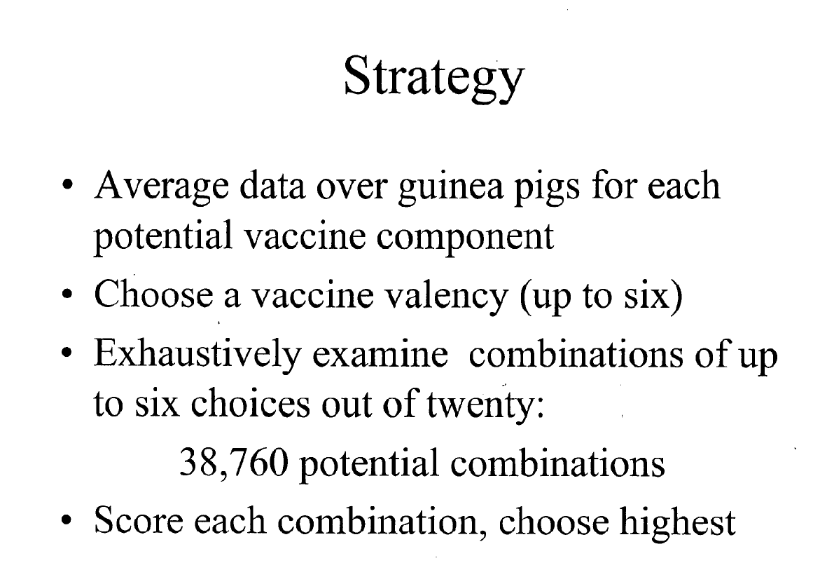

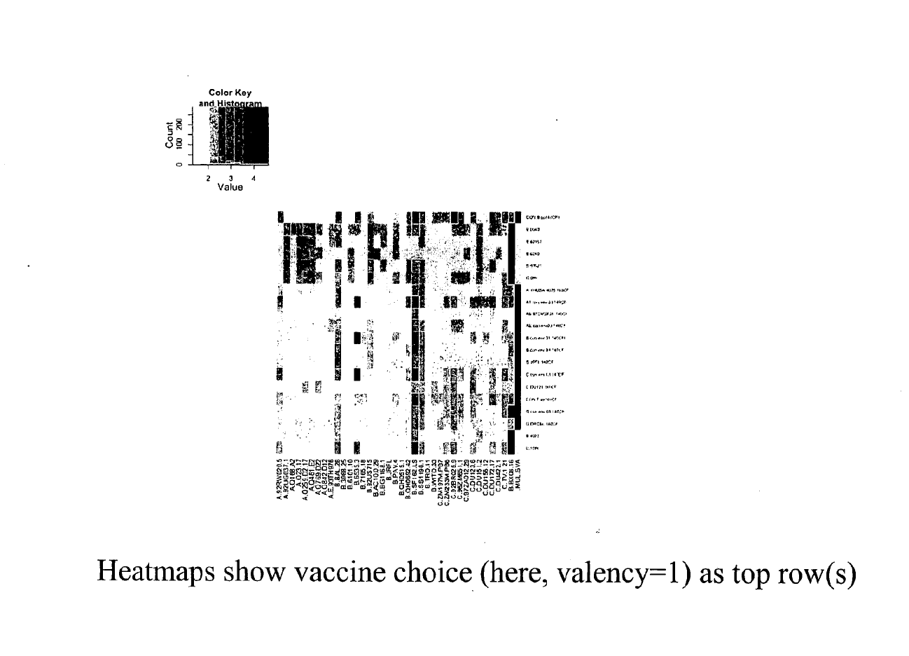

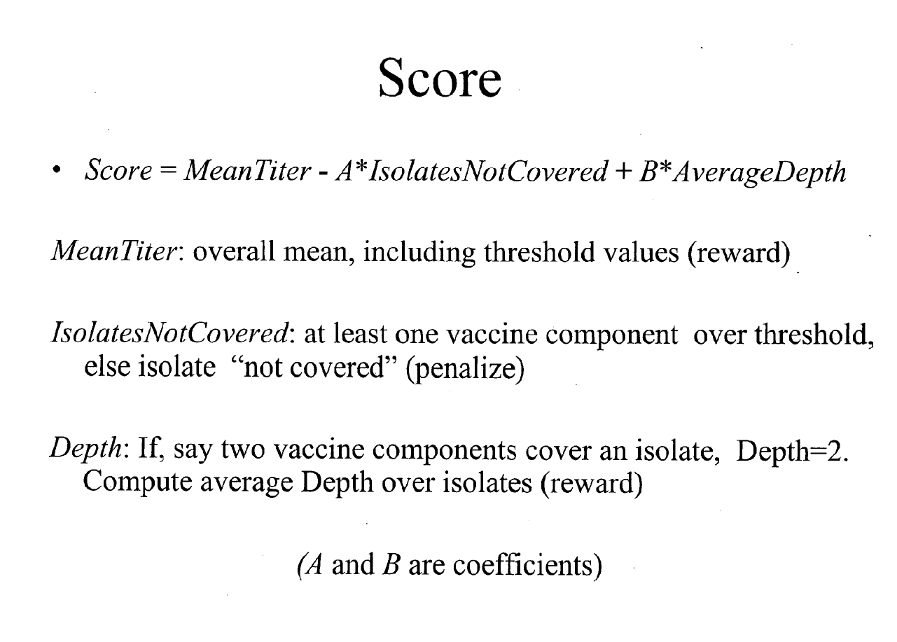

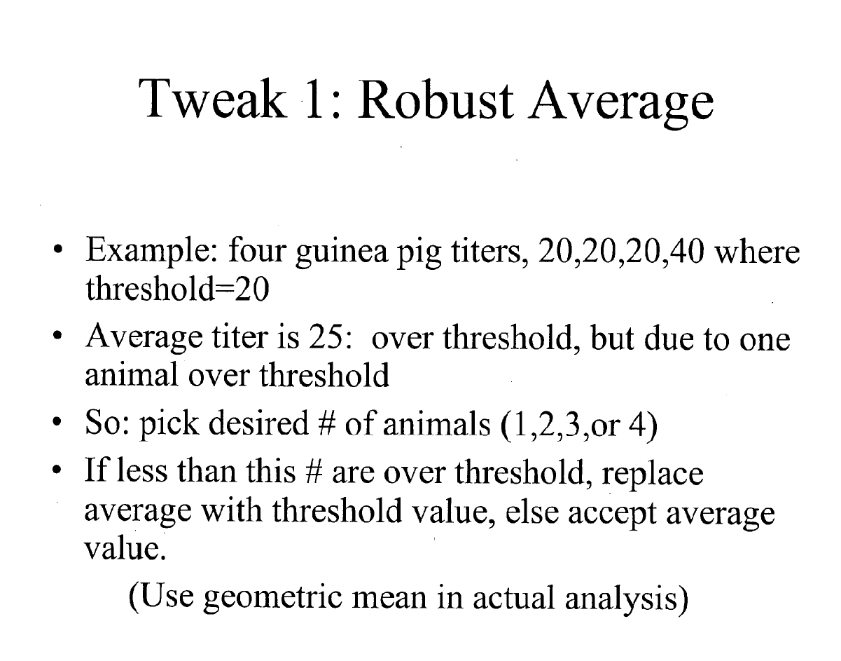

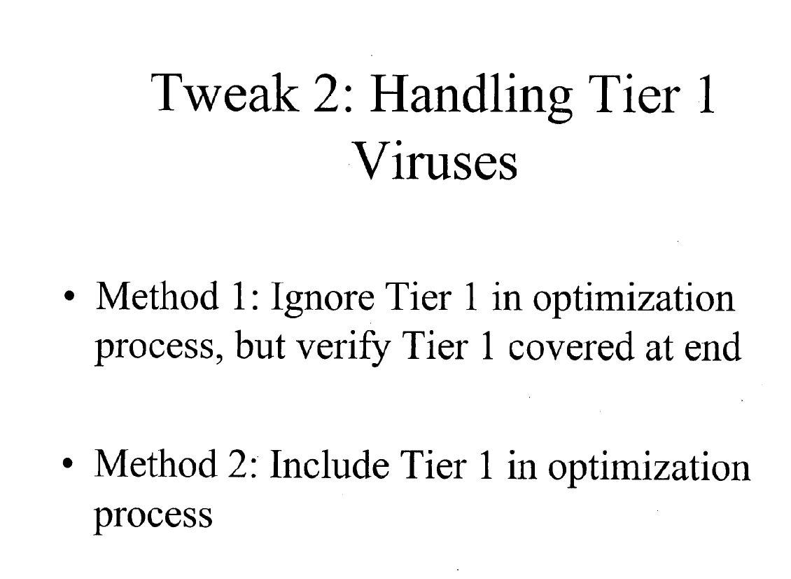

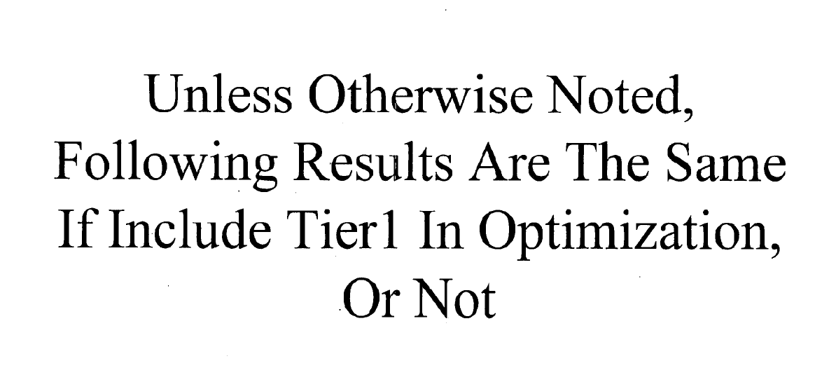

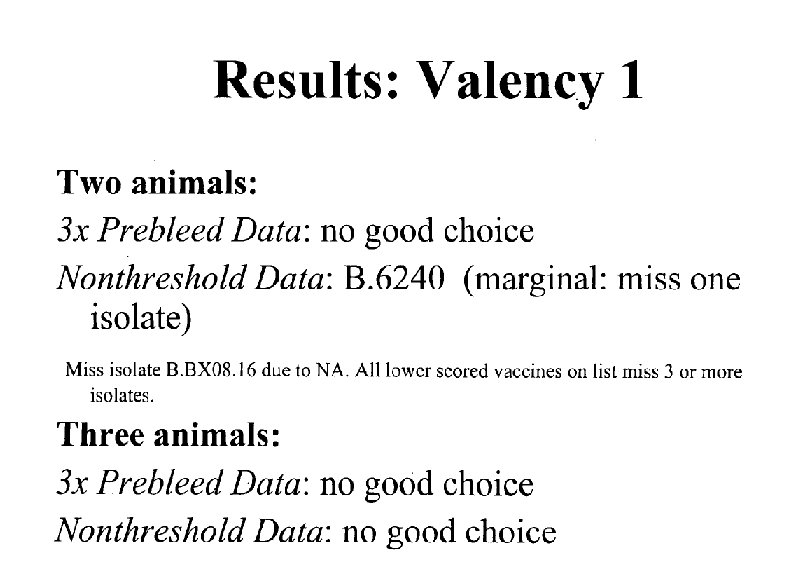

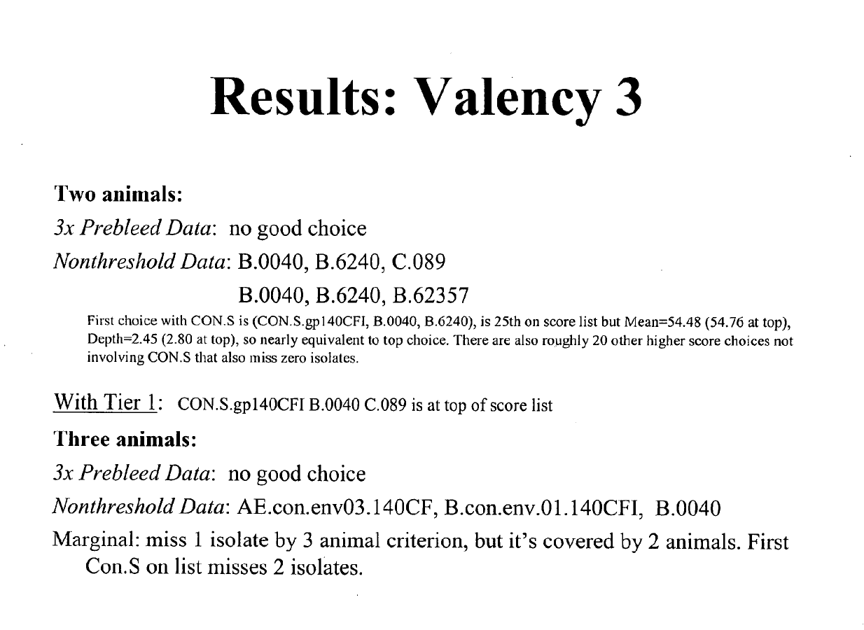

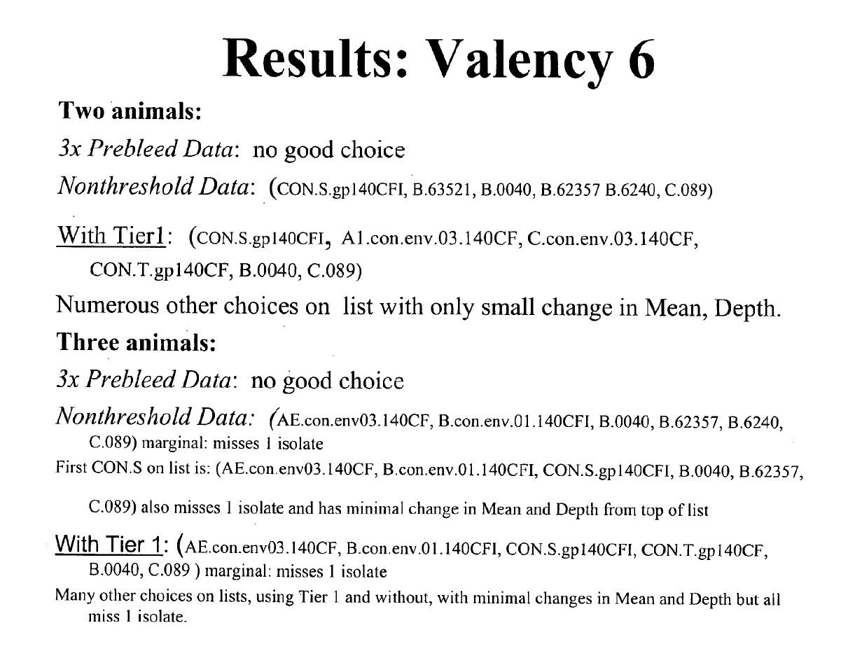

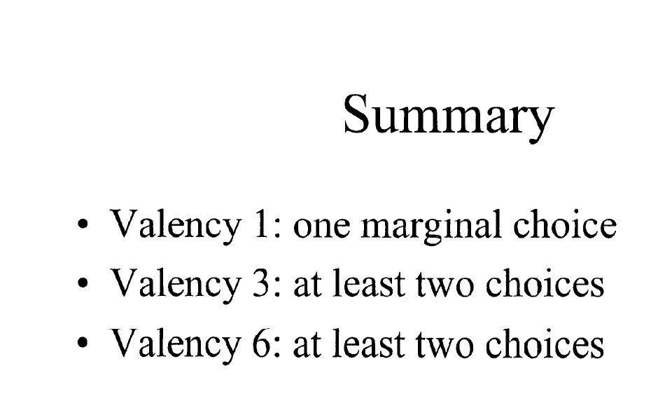

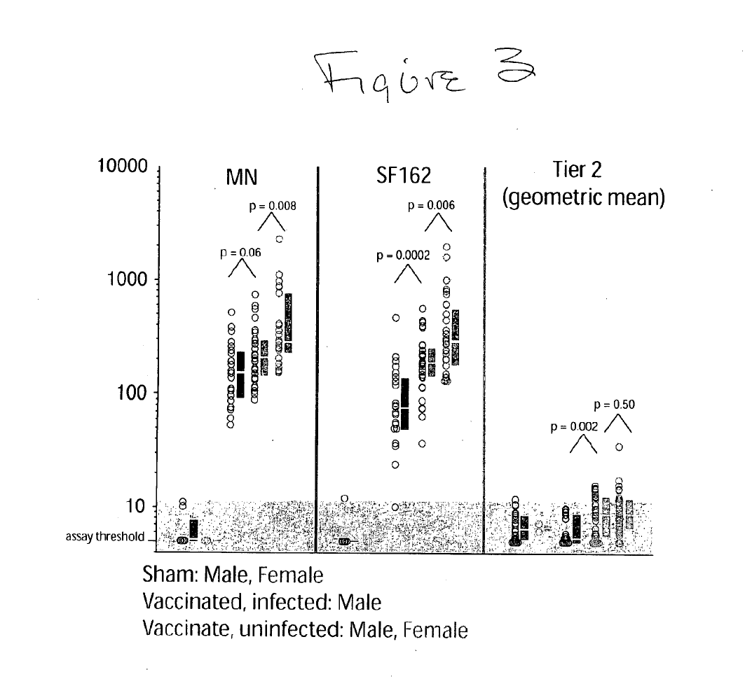

The strategy for choosing optimal polyvalent vaccines is described above and in FIG. 2.

Study # 1 considers revised HIVRAD NAb datasets with values retested according to the following:

Each pre and post serum sample was pulled and the IgG from each sample purified, and then the purified IgG was run in neutralization assays on designated isolates.

The IgG levels were brought to a concentration to be equivalent to the IgG level in serum so the titers are equivalent to what should have obtained with serum.

Those isolates were the isolates for which the prebleed was inappropriately high.

For one animal (1395) immunized with the AE 97CNGX2F gp140CF, serum was not available to purifiy the IgG so those prebleeds could not be repeated.

Animal 1395 was removed from the current analysis. The response was the observed titer value, transformed using log₁₀ titer prior to the analysis. The data contains a substantial amount of censoring due to lower and upper detection limits of the equipment. Of the 1978 data values, 900 (45.5%) were recorded as ‘<20’, 8 (0.4%) were recorded as ‘>540’, and 4 (0.2%) were recorded as ‘43,740’. Right censored data points were replaced with their lower limits (i.e., 540 and 43740, respectively). The data summary is as follows.

| Min. | 1st Qu. | Median | Mean | 3rd Qu. | Max. | NA's |
|---|---|---|---|---|---|---|
| 1.000 | 1.000 | 1.380 | 1.508 | 1.778 | 4.641 | 13.000 |

Study # 2 considers the Acute Envs (transmitter founder) Neut data. The data summary is as follows.

| Min. | 1st Qu. | Median | Mean | 3rd Qu. | Max. |
|---|---|---|---|---|---|
| 1.301 | 1.301 | 1.544 | 1.715 | 1.942 | 4.641 |

For the 20 vaccines (13 in study #1, 7 in study # 2), data were combined for the 36 envelopes which were tested simultaneously in both studies. The data for each vaccine (see Table 1) includes observed titer for (usually) four vaccinated animals whose cells are independently exposed to 37 HIV-1 strains.

TABLE 1

| Vaccine | Animal | Isolate | Titer |
|---|---|---|---|
| CONS | 1 | 1 | y11.1 |
| CONS | 1 | . | . |
| | | . | . |
| | | . | . |
| CONS | 1 | 36 | Y11.36 |
| CONS | 2 | 1 | y21.1 |
| CONS | 2 | . | . |
| | | . | . |
| | | . | . |
| CONS | 2 | 36 | Y21.36 |
| CONS | 3 | 1 | y31.1 |
| CONS | 3 | . | . |
| | | . | . |
| | | . | . |
| CONS | 3 | 36 | Y31.36 |
| CONS | 4 | 1 | y41.1 |
| CONS | 4 | . | . |
| | | . | . |
| | | . | . |
| CONS | 4 | 36 | Y41.36 |

### Generalized Linear Model

A generalized linear mixed model is used to describe a relationship between the response (log₁₀ titer) and vaccine effect. There is an interest in quantifying the effect that belonging to a particular vaccine group (relative to a reference vaccine group) has on the level of log₁₀ titer. Each guinea pig's responses to the 37 envelopes is a set of dependent responses which are influenced by the particular guinea pig's level of immune response independent of the vaccine. Animal-to-animal immune system differences are accounted for with the inclusion of individual intercepts which increase or decrease the log₁₀ titer across all isolates for a given animal.

yᵢ represents the log₁₀ titer observed (possibly censored or set equal to log₁₀ 10=1) for guinea pig i. Regression coefficient β_{k,0} represents the effect of the k-th vaccine relative to a specified reference vaccine, where β_{k,0}=0 represents no difference between the k-th vaccine and the reference vaccine, β_{k,0}>0 indicates a higher immune response for the k-th vaccine relative to the reference, and β_{k,0}<0 indicates a lower immune response for the k-th vaccine relative to the reference. Finally, Gᵢ is an intercept that augments the vaccine effect for a particular guinea pig. The model of the mean log₁₀ titer response is

E[yᵢ]=β₀+β_{1.0}Vaccine₁+β_{2.0} Vaccine₂+ . . . +β_{19.0}Vaccine₁₉+Gᵢ,

where Vaccineₖ=0 for all k if the vaccine for guinea pig i is the reference vaccine, and Vaccineₖ=1 (and all other Vaccine_{t≠k}=1) for the k-th vaccine. In the following development, models are considered with reference vaccines corresponding to those which produced the broadest responses in the two studies: CON.S.gp140CF in study # 1 and B.62357 in study # 2.

Although normally distributed response data is typically assumed in the model above, the shape of the data is markedly skewed and is not consistent with the normality assumption. The inverse Gaussian distribution has been proposed as a suitable alternative. QQ plots comparing the data to inverse Gaussian distributed data show that this distribution appears to have a superior fit. Compared to similar models fit assuming normality, the inverse Gaussian model for this data has a significantly smaller AIC and residuals which more closely follow the modeled distribution. Two alternative approaches were considered designed to handle censoring . . . namely, the ‘lmec’ (linear mixed effects model for left censored data implemented in the R ‘lmec’ package) and the ‘MCMCglmm’ algorithm (for fitting generalized linear mixed effects models for left, right, or interval censored data in the R ‘MCMCglmm’ package). The results obtained in each censored data model were consistent with those obtained in the normal fit.

### I. Set all <20s to 10

The results of a fit to the data with log10 titer response (substituting 10 for the ‘<20’ cases) assuming inverse Gaussian disturibution are given in Table 2. It includes p-values for testing a two sided hypothesis for a difference from reference group CONS (“Pval B=0”). P-values preceded with asterisks are significant at the 5% significance level. The tests for difference in vaccine effect relative to CONS under this model produced significant p-values for many vaccines, except A 1 .con.env.03.140CF, B.0040, B.62357, B.6240, B.63521, B.con.env.01.140CFI, C.089, C.con.env.03.140CF, and CON.T.gp140CF. The regression coefficients indicate that only B.0040 elicits a higher average log₁₀ titer response than CON.S.gp140CF, but this advantage is not statistically significant (p=0.57).

TABLE 2

| | Estimate | Std. Error | t value | Pval B = 0 |
|---|---|---|---|---|
| (Intercept)** | 1.800 | 0.11 | 16.00 | **1.1e−56 |
| A..00MSA.4076.140CF** | −0.520 | 0.13 | −4.10 | **4.9e−05 |
| A1.con.env.03.140CF | −0.170 | 0.15 | −1.10 | 0.26 |
| AE.97CNGX2F.140CF** | −0.550 | 0.13 | −4.10 | **3.9e−05 |
| AE.con.env03.140CF** | −0.350 | 0.14 | −2.60 | **0.01 |
| B.0040 | 0.100 | 0.18 | 0.57 | 0.57 |
| B.62357 | −0.200 | 0.14 | −1.40 | 0.17 |
| B.6240 | −0.200 | 0.14 | −1.40 | 0.16 |
| B.63521 | −0.140 | 0.15 | −0.96 | 0.34 |
| B.9021** | −0.490 | 0.13 | −3.80 | **0.00015 |
| B.con.env.01.140CFI | −0.220 | 0.14 | −1.50 | 0.14 |
| B.con.env.03.140CF** | −0.400 | 0.13 | −3.00 | **0.0029 |
| B.JRFL.140CF** | −0.410 | 0.12 | −3.30 | **0.0011 |
| C.089 | −0.053 | 0.15 | −0.35 | 0.73 |
| C.1086** | −0.290 | 0.14 | −2.10 | **0.037 |
| C.con.env.03.140CF | −0.210 | 0.14 | −1.40 | 0.15 |
| C.DU123.140CF** | −0.440 | 0.13 | −3.40 | **0.00079 |
| CON.T.gp140CF | −0.130 | 0.15 | −0.88 | 0.38 |
| G.con.env.03.140CF** | −0.400 | 0.13 | −3.00 | **0.0027 |
| G.DRCBL.140CF** | −0.390 | 0.13 | −2.90 | **0.0038 |

A similar fit with B.62357 as a reference group is summarized in Table 3. The tests for difference in vaccine effect relative to B.62357 under this model produced significant p-values (marked with asterisks) for many vaccines, except A1.con.env.03.140CF, AE.con.env03.140CF, B.0040, B.6240, B.63521, B.con.env.01.140CFI, B.con.env.03.140CF, B.JRFL.140CF, C.089, C.1086, C.con.env.03.140CF, CON.S.gp140CFI, CON.T.gp140CF, G.con.env.03.140CF, and G.DRCBL.140CF. The regression coefficients indicate that A1.con.env.03.140CF, B.0040, B.63521, C.089, CON.S.gp140CFI, and CON.T.gp140CF elicit higher average log10 titer response than B.62357 but these advantages are not statistically significant.

TABLE 3

| | Estimate | Std. Error | t value | Pval B = 0 |
|---|---|---|---|---|
| (Intercept)** | 1.6000 | 0.093 | 17.000 | **3e−63 |
| A..00MSA.4076.140CF** | −0.3200 | 0.110 | −2.800 | **0.0045 |
| A1.con.env.03.140CF | 0.0340 | 0.130 | 0.260 | 0.8 |
| AE.97CNGX2F.140CF** | −0.3500 | 0.120 | −2.900 | **0.0034 |
| AE.con.env03.140CF | −0.1500 | 0.120 | −1.200 | 0.22 |
| B.0040 | 0.3000 | 0.170 | 1.800 | 0.071 |
| B.6240 | −0.0043 | 0.130 | −0.033 | 0.97 |
| B.63521 | 0.0590 | 0.130 | 0.440 | 0.66 |
| B.9021** | −0.2900 | 0.120 | −2.500 | **0.011 |
| B.con.env.01.140CFI | −0.0150 | 0.130 | −0.120 | 0.91 |
| B.con.env.03.140CF | −0.2000 | 0.120 | −1.700 | 0.095 |
| B.JRFL.140CF | −0.2100 | 0.110 | −1.900 | 0.057 |
| C.089 | 0.1500 | 0.140 | 1.000 | 0.29 |
| C.1086 | −0.0910 | 0.130 | −0.730 | 0.47 |
| C.con.env.03.140CF | −0.0078 | 0.130 | −0.060 | 0.95 |
| C.DU123.140CF** | −0.2400 | 0.120 | −2.100 | **0.038 |
| CON.S.gp140CFI | 0.2000 | 0.140 | 1.400 | 0.17 |
| CON.T.gp140CF | 0.0700 | 0.140 | 0.520 | 0.6 |
| G.con.env.03.140CF | −0.2000 | 0.120 | −1.700 | 0.091 |
| G.DRCBL.140CF | −0.1900 | 0.120 | −1.600 | 0.11 |

### II. Set all <20s and Non-3× Prebleed to 10

Next, considered is a modified dataset in which values which were observed as ‘<20’ are set to 10, and any postbleed value which was not at least 3 times as large as the prebleed was also set to 10. When CONS is the reference vaccine group, all vaccines were found to be significantly different from CONS except for B.0040, B.6240, C.089, and C.con.env.03.140CF. The regression coefficients indicate that CONS elicits a higher response than all vaccines. (See Table 4.)

TABLE 4

| | Estimate | Std. Error | t value | Pval B = 0 |
|---|---|---|---|---|
| (Intercept)** | 1.500 | 0.086 | 18.00 | **3.6e−68 |
| A..00MSA.4076.140CF** | −0.440 | 0.100 | −4.40 | **1.1e−05 |
| A1.con.env.03.140CF** | −0.300 | 0.110 | −2.80 | **0.0046 |
| AE.97CNGX2F.140CF** | −0.480 | 0.100 | −4.60 | **4.5e−06 |
| AE.con.env03.140CF** | −0.300 | 0.110 | −2.80 | **0.0045 |
| B.0040 | −0.018 | 0.130 | −0.14 | 0.89 |
| B.62357** | −0.230 | 0.110 | −2.10 | **0.039 |
| B.6240 | −0.210 | 0.110 | −1.90 | 0.055 |
| B.63521** | −0.220 | 0.110 | −2.00 | **0.047 |
| B.9021** | −0.360 | 0.100 | −3.40 | **0.00058 |
| B.con.env.01.140CFI** | −0.370 | 0.100 | −3.50 | **4e−04 |
| B.con.env.03.140CF** | −0.300 | 0.110 | −2.80 | **0.005 |
| B.JRFL.140CF** | −0.350 | 0.097 | −3.60 | **0.00028 |
| C.089 | −0.092 | 0.120 | −0.78 | 0.43 |
| C.1086** | −0.250 | 0.110 | −2.30 | **0.022 |
| C.con.env.03.140CF | −0.210 | 0.110 | −1.90 | 0.058 |
| C.DU123.140CF** | −0.500 | 0.099 | −5.00 | **4.7e−07 |
| CON.T.gp140CF** | −0.270 | 0.110 | −2.50 | **0.012 |
| G.con.env.03.140CF** | −0.300 | 0.110 | −2.90 | **0.0043 |
| G.DRCBL.140CF** | −0.380 | 0.100 | −3.60 | **0.00028 |

With B.62357 as a reference vaccine, it is found that most vaccines do not have a significantly different effect from B.62357, including A1.con.env.03.140CF, AE.con.env03.140CF, B.0040, B.6240, B.63521, B.9021, B.con.env.01.140CFI, B.con.env.03.140CF, B.JRFL.140CF, C.089, C.1086, C.con.env.03.140CF, CON.T.gp140CF, G.con.env.03.140CF, G.DRCBL.140CF. The regression coefficients indicate that B.0040, B.6240, B.63521, C.089, C.con.env.03.140CF, and CONS elicit higher average log 10 titer response than CONS but this advantage is not statistically significant. (See Table 5.)

TABLE 5

| | Estimate | Std. Error | t value | Pval B = 0 |
|---|---|---|---|---|
| (Intercept)** | 1.3000 | 0.068 | 19.000 | **6.8e−79 |
| A..00MSA.4076.140CF** | −0.2200 | 0.086 | −2.500 | **0.012 |
| A1.con.env.03.140CF | −0.0760 | 0.093 | −0.830 | 0.41 |
| AE.97CNGX2F.140CF** | −0.2500 | 0.089 | −2.800 | **0.0051 |
| AE.con.env03.140CF | −0.0760 | 0.092 | −0.820 | 0.41 |
| B.0040 | 0.2100 | 0.120 | 1.800 | 0.08 |
| B.6240 | 0.0150 | 0.097 | 0.150 | 0.88 |
| B.63521 | 0.0076 | 0.096 | 0.079 | 0.94 |
| B.9021 | −0.1300 | 0.089 | −1.500 | 0.14 |
| B.con.env.01.140CFI | −0.1400 | 0.089 | −1.600 | 0.11 |
| B.con.env.03.140CF | −0.0720 | 0.092 | −0.780 | 0.44 |
| B.JRFL.140CF | −0.1300 | 0.081 | −1.600 | 0.12 |
| C.089 | 0.1400 | 0.100 | 1.300 | 0.19 |
| C.1086 | −0.0230 | 0.095 | −0.240 | 0.81 |
| C.con.env.03.140CF | 0.0160 | 0.098 | 0.170 | 0.87 |
| C.DU123.140CF** | −0.2700 | 0.083 | −3.300 | **0.0011 |
| CON.S.gp140CFI** | 0.2300 | 0.110 | 2.100 | **0.039 |
| CON.T.gp140CF | −0.0440 | 0.094 | −0.470 | 0.64 |
| G.con.env.03.140CF | −0.0770 | 0.092 | −0.840 | 0.4 |
| G.DRCBL.140CF | −0.1500 | 0.089 | −1.700 | 0.093 |

In summary, two versions of the combined dataset were considered. In the first version, all <20 values were replaced with 10. The second version used 10 in place of all <20 observations and those postbleed values which were not at least 3 times the prebleed value. The analyses of two data versions indicated similar numbers of vaccine differences.

The replacement of values which did not meet the threshold had a significant effect on the determination of which vaccines differed from CONS. If the 3× positivity criteria are ignored, it is found that the following vaccines were not significantly different from CONS: A1.con.env.03.140CF, B.0040, B.62357, B.6240, B.63521, B.con.env.01.140CFI, C.089, C.con.env.03.140CF, and CON.T.gp140CF. When the 3× positivity criteria is considered, it is found that the following vaccines were not significantly different from CONS: B.0040, B.6240, C.089, and C.con.env.03.140CF. A systematic increase or decrease in effect is not found using either criteria but the more stringent criteria appears to accentuate the number of significant differences from CONS.

When the 3× threshold is not considered, it is found that the following vaccines were not significantly different from B.62357: A1.con.env.03.140CF, AE.con.env03.140CF, B.0040, B.6240, B.63521; B.con.env.01.140CFI, B.con.env.03.140CF, B.JRFL.140CF, C.089, C.1086, C.con.env.03.140CF, CON.S.gp140CFI, CON.T.gp140CF, G.con.env.03.140CF, and G.DRCBL.140CF. With the 3× threshold, it is found that the following vaccines were not significantly different from B.62357: A1.con.env.03.140CF, AE.con.env03.140CF, B.0040, B.6240, B.63521, B.9021, B.con.env.01.140CFI, B.con.env.03.140CF, B.JRFL.140CF, C.089, C.1086, C.con.env.03.140CF, CON.T.gp140CF, G.con.env.03.140CF, G.DRCBL.140CF. Again, there is no evidence of a systematic increase or decrease in effect for either criteria but these models roughly indicate that the same set of vaccines significantly differ from B.62357.

The regression coefficients in the GLM analyses indicate the augmentation of the CONS or B.63257 vaccine effect (summarized by the intercept term). The regression coefficient for B.0040 is highest in magnitude among those with positive coefficient estimates in all four models. Thus, it is found that vaccine B.0040 produces the highest overall vaccine effect.

All documents and other information sources cited above are hereby incorporated in their entirety by reference.

1. A polyvalent immunogen comprising gp140 Envs:
i) B.0040, B.6240 and C.089,
ii) B.0040, B.6240 and B.62357,
iii) CONS, B.0040 and B.6240, or
iv) CON.S, 8.0040 and C.089.

2. A polyvalent immunogen comprising gp140 ENVS:
i) CON.S, B.63521, B.0040, B.62357, B.6240, and C.089, or
ii) CON.S, A1.con.env.03.140CF, C.con.env.03.140CF, CON.T, 8.0040 and C.089.

3. A composition comprising the polyvalent immunogen of claim 1 and an adjuvant.

4. A method of inducing the production in a subject of an immune response against HIV-1 comprising administering to said subject the polyvalent immunogen of claim 1 in an amount sufficient to effect said induction.

5. The method according to claim 4 further comprising administering to said subject an adjuvant.

6. The method according to claim 5 wherein said adjuvant is monophosphorylipid A, a TLR9 agonist, or a squalene-based adjuvant.

7. The method according to claim 4 wherein said polyvalent immunogen is produced in said subject following introduction into said subject of a nucleotide sequence, or nucleotide sequences, encoding said polyvalent immunogen under conditions such that said nucleotide sequence, or nucleotide sequences, is/are expressed and production of said polyvalent immunogen is thereby effected,

8. The method according to claim 4 wherein said subject is a human.

9. Use of a polyvalent immunogen comprising gp140 Envs:
i) B.0040, B.6240 and 0.089,
ii) B.0040, B.6240 and B.62357.
iii) CON.S, B.0040 and B.6240, or
iv) CONS, B.0040 and C.089,
or nucleotide sequence, or nucleotide sequences, encoding said polyvalent immunogen,
for inducing an immune response against HIV-1 in a subject.

10. Use of a polyvalent immunogen comprising gp140 Envs:
i) CON.S, B.63521, B.0040, B.62357, B.6240, and C.089, or
ii) CON.S, Al .con.env.03.140CF, C.con.env.03.140CF, CON.T, 8.0040 and C.089,
or nucleotide sequence, or nucleotide sequences, encoding said polyvalent immunogen,
for inducing an immune response against HIV-1 in a subject.

11. A composition comprising the polyvalent immunogen of claim 2 and an adjuvant.

12. A method of inducing the production in a subject of an immune response against HIV-1 comprising administering to said subject the polyvalent immunogen of claim 2 in an amount sufficient to effect said induction.

13. The method according to claim 12 further comprising administering to said subject an adjuvant.

14. The method according to claim 13 wherein said adjuvant is monophosphorylipid A, a TLR9 agonist, or a squalene-based adjuvant.

15. The method according to claim 12 wherein said polyvalent immunogen is produced in said subject following introduction into said subject of a nucleotide sequence, or nucleotide sequences, encoding said polyvalent immunogen under conditions such that said nucleotide sequence, or nucleotide sequences, is/are expressed and production of said polyvalent immunogen is thereby effected.

16. The method according to claim 12 wherein said subject is a human.
